SEQUENCE LISTING (217 sequences)

SEQ ID NO: 1 - DNA, 36 bp, Artificial Sequence
  source: /note=“Description of Artificial Sequence Synthetic oligonucleotide”
tctttccctacacgacgctcttccgatctcagatct

SEQ ID NO: 2 - DNA, 34 bp, Artificial Sequence
  source: /note=“Description of Artificial Sequence Synthetic oligonucleotide”
agatcggaagagcacacgtctgaactccagtcac

SEQ ID NO: 3 - DNA, 36 bp, Artificial Sequence; identical to SEQ ID NO: 1
  source: /note=“Description of Artificial Sequence Synthetic oligonucleotide”

SEQ ID NO: 4 - DNA, 40 bp, Artificial Sequence
  source: /note=“Description of Artificial Sequence Synthetic oligonucleotide”
gatctgagatcggaagagcacacgtctgaactccagtcac

SEQ ID NO: 5 - DNA, 62 bp, Artificial Sequence
  source: /note=“Description of Artificial Sequence Synthetic oligonucleotide”
aatgatacggcgaccaccgagatctacacgttaagcgacactctttccctacacgacgctct

SEQ ID NO: 6 - DNA, 62 bp, Artificial Sequence
  source: /note=“Description of Artificial Sequence Synthetic oligonucleotide”
aatgatacggcgaccaccgagatctacacgagaccaaacactctttccctacacgacgctct

SEQ ID NO: 7 - DNA, 62 bp, Artificial Sequence
  source: /note=“Description of Artificial Sequence Synthetic oligonucleotide”
aatgatacggcgaccaccgagatctacacagccgtaaacactctttccctacacgacgctct

SEQ ID NO: 8 - DNA, 62 bp, Artificial Sequence
  source: /note=“Description of Artificial Sequence Synthetic oligonucleotide”
aatgatacggcgaccaccgagatctacacttcgaagcacactctttccctacacgacgctct

SEQ ID NO: 9 - DNA, 62 bp, Artificial Sequence
  source: /note=“Description of Artificial Sequence Synthetic oligonucleotide”
aatgatacggcgaccaccgagatctacacatgacaggacactctttccctacacgacgctct

SEQ ID NO: 10 - DNA, 62 bp, Artificial Sequence
  source: /note=“Description of Artificial Sequence Synthetic oligonucleotide”
aatgatacggcgaccaccgagatctacactcgtgcatacactctttccctacacgacgctct

SEQ ID NO: 11 - DNA, 62 bp, Artificial Sequence
  source: /note=“Description of Artificial Sequence Synthetic oligonucleotide”
aatgatacggcgaccaccgagatctacaccgaagtcaacactctttccctacacgacgctct

SEQ ID NO: 12 - DNA, 62 bp, Artificial Sequence
  source: /note=“Description of Artificial Sequence Synthetic oligonucleotide”
aatgatacggcgaccaccgagatctacacgaatccgtacactctttccctacacgacgctct

SEQ ID NO: 13 - DNA, 62 bp, Artificial Sequence
  source: /note=“Description of Artificial Sequence Synthetic oligonucleotide”
aatgatacggcgaccaccgagatctacacgaagtgctacactctttccctacacgacgctct

SEQ ID NO: 14 - DNA, 62 bp, Artificial Sequence
  source: /note=“Description of Artificial Sequence Synthetic oligonucleotide”
aatgatacggcgaccaccgagatctacacgtccttgaacactctttccctacacgacgctct

SEQ ID NO: 15 - DNA, 62 bp, Artificial Sequence
  source: /note=“Description of Artificial Sequence Synthetic oligonucleotide”
aatgatacggcgaccaccgagatctacaccatgtgtgacactctttccctacacgacgctct

SEQ ID NO: 16 - DNA, 62 bp, Artificial Sequence
  source: /note=“Description of Artificial Sequence Synthetic oligonucleotide”
aatgatacggcgaccaccgagatctacacacctcttcacactctttccctacacgacgctct

SEQ ID NO: 17 - DNA, 62 bp, Artificial Sequence
  source: /note=“Description of Artificial Sequence Synthetic oligonucleotide”
aatgatacggcgaccaccgagatctacactccgatcaacactctttccctacacgacgctct

SEQ ID NO: 18 - DNA, 62 bp, Artificial Sequence
  source: /note=“Description of Artificial Sequence Synthetic oligonucleotide”
aatgatacggcgaccaccgagatctacaccgtatctcacactctttccctacacgacgctct

SEQ ID NO: 19 - DNA, 62 bp, Artificial Sequence
  source: /note=“Description of Artificial Sequence Synthetic oligonucleotide”
aatgatacggcgaccaccgagatctacacttgcaacgacactctttccctacacgacgctct

SEQ ID NO: 20 - DNA, 62 bp, Artificial Sequence
  source: /note=“Description of Artificial Sequence Synthetic oligonucleotide”
aatgatacggcgaccaccgagatctacactgataggcacactctttccctacacgacgctct

SEQ ID NO: 21 - DNA, 62 bp, Artificial Sequence
  source: /note=“Description of Artificial Sequence Synthetic oligonucleotide”
aatgatacggcgaccaccgagatctacacaacagtccacactctttccctacacgacgctct

SEQ ID NO: 22 - DNA, 62 bp, Artificial Sequence
  source: /note=“Description of Artificial Sequence Synthetic oligonucleotide”
aatgatacggcgaccaccgagatctacacaggaacacacactctttccctacacgacgctct

SEQ ID NO: 23 - DNA, 62 bp, Artificial Sequence
  source: /note=“Description of Artificial Sequence Synthetic oligonucleotide”
aatgatacggcgaccaccgagatctacactcctcatgacactctttccctacacgacgctct

SEQ ID NO: 24 - DNA, 62 bp, Artificial Sequence
  source: /note=“Description of Artificial Sequence Synthetic oligonucleotide”
aatgatacggcgaccaccgagatctacacagagcagaacactctttccctacacgacgctct

SEQ ID NO: 25 - DNA, 62 bp, Artificial Sequence
  source: /note=“Description of Artificial Sequence Synthetic oligonucleotide”
aatgatacggcgaccaccgagatctacacgaacgaagacactctttccctacacgacgctct

SEQ ID NO: 26 - DNA, 62 bp, Artificial Sequence
  source: /note=“Description of Artificial Sequence Synthetic oligonucleotide”
aatgatacggcgaccaccgagatctacacttgagctcacactctttccctacacgacgctct

SEQ ID NO: 27 - DNA, 62 bp, Artificial Sequence
  source: /note=“Description of Artificial Sequence Synthetic oligonucleotide”
aatgatacggcgaccaccgagatctacacgctgaatcacactctttccctacacgacgctct

SEQ ID NO: 28 - DNA, 62 bp, Artificial Sequence
  source: /note=“Description of Artificial Sequence Synthetic oligonucleotide”
aatgatacggcgaccaccgagatctacacagattgcgacactctttccctacacgacgctct

SEQ ID NO: 29 - DNA, 62 bp, Artificial Sequence
  source: /note=“Description of Artificial Sequence Synthetic oligonucleotide”
aatgatacggcgaccaccgagatctacaccaacttggacactctttccctacacgacgctct

SEQ ID NO: 30 - DNA, 62 bp, Artificial Sequence
  source: /note=“Description of Artificial Sequence Synthetic oligonucleotide”
aatgatacggcgaccaccgagatctacacttggtgcaacactctttccctacacgacgctct

SEQ ID NO: 31 - DNA, 62 bp, Artificial Sequence
  source: /note=“Description of Artificial Sequence Synthetic oligonucleotide”
aatgatacggcgaccaccgagatctacacctgtaccaacactctttccctacacgacgctct

SEQ ID NO: 32 - DNA, 62 bp, Artificial Sequence
  source: /note=“Description of Artificial Sequence Synthetic oligonucleotide”
aatgatacggcgaccaccgagatctacacactctgagacactctttccctacacgacgctct

SEQ ID NO: 33 - DNA, 62 bp, Artificial Sequence
  source: /note=“Description of Artificial Sequence Synthetic oligonucleotide”
aatgatacggcgaccaccgagatctacacctcctagtacactctttccctacacgacgctct

SEQ ID NO: 34 - DNA, 62 bp, Artificial Sequence
  source: /note=“Description of Artificial Sequence Synthetic oligonucleotide”
aatgatacggcgaccaccgagatctacacgccaatacacactctttccctacacgacgctct

SEQ ID NO: 35 - DNA, 62 bp, Artificial Sequence
  source: /note=“Description of Artificial Sequence Synthetic oligonucleotide”
aatgatacggcgaccaccgagatctacaccctcatctacactctttccctacacgacgctct

SEQ ID NO: 36 - DNA, 62 bp, Artificial Sequence
  source: /note=“Description of Artificial Sequence Synthetic oligonucleotide”
aatgatacggcgaccaccgagatctacactgagctgtacactctttccctacacgacgctct

SEQ ID NO: 37 - DNA, 62 bp, Artificial Sequence
  source: /note=“Description of Artificial Sequence Synthetic oligonucleotide”
aatgatacggcgaccaccgagatctacacgtctcatcacactctttccctacacgacgctct

SEQ ID NO: 38 - DNA, 62 bp, Artificial Sequence
  source: /note=“Description of Artificial Sequence Synthetic oligonucleotide”
aatgatacggcgaccaccgagatctacactaagcgcaacactctttccctacacgacgctct

SEQ ID NO: 39 - DNA, 62 bp, Artificial Sequence
  source: /note=“Description of Artificial Sequence Synthetic oligonucleotide”
aatgatacggcgaccaccgagatctacacagctaccaacactctttccctacacgacgctct

SEQ ID NO: 40 - DNA, 62 bp, Artificial Sequence
  source: /note=“Description of Artificial Sequence Synthetic oligonucleotide”
aatgatacggcgaccaccgagatctacaccttcactgacactctttccctacacgacgctct

SEQ ID NO: 41 - DNA, 62 bp, Artificial Sequence
  source: /note=“Description of Artificial Sequence Synthetic oligonucleotide”
aatgatacggcgaccaccgagatctacacgagagtacacactctttccctacacgacgctct

SEQ ID NO: 42 - DNA, 62 bp, Artificial Sequence
  source: /note=“Description of Artificial Sequence Synthetic oligonucleotide”
aatgatacggcgaccaccgagatctacacgcgttagaacactctttccctacacgacgctct

SEQ ID NO: 43 - DNA, 62 bp, Artificial Sequence
  source: /note=“Description of Artificial Sequence Synthetic oligonucleotide”
aatgatacggcgaccaccgagatctacacaggcaatgacactctttccctacacgacgctct

SEQ ID NO: 44 - DNA, 62 bp, Artificial Sequence
  source: /note=“Description of Artificial Sequence Synthetic oligonucleotide”
aatgatacggcgaccaccgagatctacacgctacaacacactctttccctacacgacgctct

SEQ ID NO: 45 - DNA, 62 bp, Artificial Sequence
  source: /note=“Description of Artificial Sequence Synthetic oligonucleotide”
aatgatacggcgaccaccgagatctacactcagtaggacactctttccctacacgacgctct

SEQ ID NO: 46 - DNA, 62 bp, Artificial Sequence
  source: /note=“Description of Artificial Sequence Synthetic oligonucleotide”
aatgatacggcgaccaccgagatctacacctatgcctacactctttccctacacgacgctct

SEQ ID NO: 47 - DNA, 62 bp, Artificial Sequence
  source: /note=“Description of Artificial Sequence Synthetic oligonucleotide”
aatgatacggcgaccaccgagatctacactgctgtgaacactctttccctacacgacgctct

SEQ ID NO: 48 - DNA, 62 bp, Artificial Sequence
  source: /note=“Description of Artificial Sequence Synthetic oligonucleotide”
aatgatacggcgaccaccgagatctacacccgaagatacactctttccctacacgacgctct

SEQ ID NO: 49 - DNA, 62 bp, Artificial Sequence
  source: /note=“Description of Artificial Sequence Synthetic oligonucleotide”
aatgatacggcgaccaccgagatctacacagaccttgacactctttccctacacgacgctct

SEQ ID NO: 50 - DNA, 62 bp, Artificial Sequence
  source: /note=“Description of Artificial Sequence Synthetic oligonucleotide”
aatgatacggcgaccaccgagatctacacactgcttgacactctttccctacacgacgctct

SEQ ID NO: 51 - DNA, 62 bp, Artificial Sequence
  source: /note=“Description of Artificial Sequence Synthetic oligonucleotide”
aatgatacggcgaccaccgagatctacactaagtggcacactctttccctacacgacgctct

SEQ ID NO: 52 - DNA, 62 bp, Artificial Sequence
  source: /note=“Description of Artificial Sequence Synthetic oligonucleotide”
aatgatacggcgaccaccgagatctacaccgcaatgtacactctttccctacacgacgctct

SEQ ID NO: 53 - DNA, 62 bp, Artificial Sequence
  source: /note=“Description of Artificial Sequence Synthetic oligonucleotide”
aatgatacggcgaccaccgagatctacactgaccgttacactctttccctacacgacgctct

SEQ ID NO: 54 - DNA, 62 bp, Artificial Sequence
  source: /note=“Description of Artificial Sequence Synthetic oligonucleotide”
aatgatacggcgaccaccgagatctacaccctcgaatacactctttccctacacgacgctct

SEQ ID NO: 55 - DNA, 62 bp, Artificial Sequence
  source: /note=“Description of Artificial Sequence Synthetic oligonucleotide”
aatgatacggcgaccaccgagatctacactgctctacacactctttccctacacgacgctct

SEQ ID NO: 56 - DNA, 62 bp, Artificial Sequence
  source: /note=“Description of Artificial Sequence Synthetic oligonucleotide”
aatgatacggcgaccaccgagatctacacgtcgttacacactctttccctacacgacgctct

SEQ ID NO: 57 - DNA, 62 bp, Artificial Sequence
  source: /note=“Description of Artificial Sequence Synthetic oligonucleotide”
aatgatacggcgaccaccgagatctacacatagtcggacactctttccctacacgacgctct

SEQ ID NO: 58 - DNA, 62 bp, Artificial Sequence
  source: /note=“Description of Artificial Sequence Synthetic oligonucleotide”
aatgatacggcgaccaccgagatctacactagcaggaacactctttccctacacgacgctct

SEQ ID NO: 59 - DNA, 62 bp, Artificial Sequence
  source: /note=“Description of Artificial Sequence Synthetic oligonucleotide”
aatgatacggcgaccaccgagatctacactacggaagacactctttccctacacgacgctct

SEQ ID NO: 60 - DNA, 62 bp, Artificial Sequence
  source: /note=“Description of Artificial Sequence Synthetic oligonucleotide”
aatgatacggcgaccaccgagatctacacaggtgttgacactctttccctacacgacgctct

SEQ ID NO: 61 - DNA, 62 bp, Artificial Sequence
  source: /note=“Description of Artificial Sequence Synthetic oligonucleotide”
aatgatacggcgaccaccgagatctacacccgatgtaacactctttccctacacgacgctct

SEQ ID NO: 62 - DNA, 62 bp, Artificial Sequence
  source: /note=“Description of Artificial Sequence Synthetic oligonucleotide”
aatgatacggcgaccaccgagatctacacctcgacttacactctttccctacacgacgctct

SEQ ID NO: 63 - DNA, 62 bp, Artificial Sequence
  source: /note=“Description of Artificial Sequence Synthetic oligonucleotide”
aatgatacggcgaccaccgagatctacacgtagtaccacactctttccctacacgacgctct

SEQ ID NO: 64 - DNA, 62 bp, Artificial Sequence
  source: /note=“Description of Artificial Sequence Synthetic oligonucleotide”
aatgatacggcgaccaccgagatctacacattagccgacactctttccctacacgacgctct

SEQ ID NO: 65 - DNA, 62 bp, Artificial Sequence
  source: /note=“Description of Artificial Sequence Synthetic oligonucleotide”
aatgatacggcgaccaccgagatctacactggaccatacactctttccctacacgacgctct

SEQ ID NO: 66 - DNA, 62 bp, Artificial Sequence
  source: /note=“Description of Artificial Sequence Synthetic oligonucleotide”
aatgatacggcgaccaccgagatctacaccatctgctacactctttccctacacgacgctct

SEQ ID NO: 67 - DNA, 62 bp, Artificial Sequence
  source: /note=“Description of Artificial Sequence Synthetic oligonucleotide”
aatgatacggcgaccaccgagatctacacgactacgaacactctttccctacacgacgctct

SEQ ID NO: 68 - DNA, 62 bp, Artificial Sequence
  source: /note=“Description of Artificial Sequence Synthetic oligonucleotide”
aatgatacggcgaccaccgagatctacacgcttcacaacactctttccctacacgacgctct

SEQ ID NO: 69 - DNA, 62 bp, Artificial Sequence
  source: /note=“Description of Artificial Sequence Synthetic oligonucleotide”
aatgatacggcgaccaccgagatctacacaacgtagcacactctttccctacacgacgctct

SEQ ID NO: 70 - DNA, 62 bp, Artificial Sequence
  source: /note=“Description of Artificial Sequence Synthetic oligonucleotide”
aatgatacggcgaccaccgagatctacacaccatgtcacactctttccctacacgacgctct

SEQ ID NO: 71 - DNA, 62 bp, Artificial Sequence
  source: /note=“Description of Artificial Sequence Synthetic oligonucleotide”
aatgatacggcgaccaccgagatctacacctgtggtaacactctttccctacacgacgctct

SEQ ID NO: 72 - DNA, 62 bp, Artificial Sequence
  source: /note=“Description of Artificial Sequence Synthetic oligonucleotide”
aatgatacggcgaccaccgagatctacacgttggcatacactctttccctacacgacgctct

SEQ ID NO: 73 - DNA, 62 bp, Artificial Sequence
  source: /note=“Description of Artificial Sequence Synthetic oligonucleotide”
aatgatacggcgaccaccgagatctacacgatacctgacactctttccctacacgacgctct

SEQ ID NO: 74 - DNA, 62 bp, Artificial Sequence
  source: /note=“Description of Artificial Sequence Synthetic oligonucleotide”
aatgatacggcgaccaccgagatctacacgacgtcatacactctttccctacacgacgctct

SEQ ID NO: 75 - DNA, 62 bp, Artificial Sequence
  source: /note=“Description of Artificial Sequence Synthetic oligonucleotide”
aatgatacggcgaccaccgagatctacaccaggatgtacactctttccctacacgacgctct

SEQ ID NO: 76 - DNA, 62 bp, Artificial Sequence
  source: /note=“Description of Artificial Sequence Synthetic oligonucleotide”
aatgatacggcgaccaccgagatctacacacaccgatacactctttccctacacgacgctct

SEQ ID NO: 77 - DNA, 62 bp, Artificial Sequence
  source: /note=“Description of Artificial Sequence Synthetic oligonucleotide”
aatgatacggcgaccaccgagatctacactgcttgctacactctttccctacacgacgctct

SEQ ID NO: 78 - DNA, 62 bp, Artificial Sequence
  source: /note=“Description of Artificial Sequence Synthetic oligonucleotide”
aatgatacggcgaccaccgagatctacactggaagcaacactctttccctacacgacgctct

SEQ ID NO: 79 - DNA, 62 bp, Artificial Sequence
  source: /note=“Description of Artificial Sequence Synthetic oligonucleotide”
aatgatacggcgaccaccgagatctacactatgaccgacactctttccctacacgacgctct

SEQ ID NO: 80 - DNA, 62 bp, Artificial Sequence
  source: /note=“Description of Artificial Sequence Synthetic oligonucleotide”
aatgatacggcgaccaccgagatctacacccgcttaaacactctttccctacacgacgctct

SEQ ID NO: 81 - DNA, 62 bp, Artificial Sequence
  source: /note=“Description of Artificial Sequence Synthetic oligonucleotide”
aatgatacggcgaccaccgagatctacaccctcgttaacactctttccctacacgacgctct

SEQ ID NO: 82 - DNA, 62 bp, Artificial Sequence
  source: /note=“Description of Artificial Sequence Synthetic oligonucleotide”
aatgatacggcgaccaccgagatctacacagctaagcacactctttccctacacgacgctct

SEQ ID NO: 83 - DNA, 62 bp, Artificial Sequence
  source: /note=“Description of Artificial Sequence Synthetic oligonucleotide”
aatgatacggcgaccaccgagatctacacctaagaccacactctttccctacacgacgctct

SEQ ID NO: 84 - DNA, 62 bp, Artificial Sequence
  source: /note=“Description of Artificial Sequence Synthetic oligonucleotide”
aatgatacggcgaccaccgagatctacactcacctagacactctttccctacacgacgctct

SEQ ID NO: 85 - DNA, 62 bp, Artificial Sequence
  source: /note=“Description of Artificial Sequence Synthetic oligonucleotide”
aatgatacggcgaccaccgagatctacacgcataacgacactctttccctacacgacgctct

SEQ ID NO: 86 - DNA, 62 bp, Artificial Sequence
  source: /note=“Description of Artificial Sequence Synthetic oligonucleotide”
aatgatacggcgaccaccgagatctacacaggttcctacactctttccctacacgacgctct

SEQ ID NO: 87 - DNA, 62 bp, Artificial Sequence
  source: /note=“Description of Artificial Sequence Synthetic oligonucleotide”
aatgatacggcgaccaccgagatctacaccgagttagacactctttccctacacgacgctct

SEQ ID NO: 88 - DNA, 62 bp, Artificial Sequence
  source: /note=“Description of Artificial Sequence Synthetic oligonucleotide”
aatgatacggcgaccaccgagatctacactcttcgacacactctttccctacacgacgctct

SEQ ID NO: 89 - DNA, 62 bp, Artificial Sequence
  source: /note=“Description of Artificial Sequence Synthetic oligonucleotide”
aatgatacggcgaccaccgagatctacactactgctcacactctttccctacacgacgctct

SEQ ID NO: 90 - DNA, 62 bp, Artificial Sequence
  source: /note=“Description of Artificial Sequence Synthetic oligonucleotide”
aatgatacggcgaccaccgagatctacacctgccataacactctttccctacacgacgctct

SEQ ID NO: 91 - DNA, 62 bp, Artificial Sequence
  source: /note=“Description of Artificial Sequence Synthetic oligonucleotide”
aatgatacggcgaccaccgagatctacacccaagtagacactctttccctacacgacgctct

SEQ ID NO: 92 - DNA, 62 bp, Artificial Sequence
  source: /note=“Description of Artificial Sequence Synthetic oligonucleotide”
aatgatacggcgaccaccgagatctacacgaccgataacactctttccctacacgacgctct

SEQ ID NO: 93 - DNA, 62 bp, Artificial Sequence
  source: /note=“Description of Artificial Sequence Synthetic oligonucleotide”
aatgatacggcgaccaccgagatctacaccatacggaacactctttccctacacgacgctct

SEQ ID NO: 94 - DNA, 62 bp, Artificial Sequence
  source: /note=“Description of Artificial Sequence Synthetic oligonucleotide”
aatgatacggcgaccaccgagatctacactctagtccacactctttccctacacgacgctct

SEQ ID NO: 95 - DNA, 62 bp, Artificial Sequence
  source: /note=“Description of Artificial Sequence Synthetic oligonucleotide”
aatgatacggcgaccaccgagatctacacagtgacctacactctttccctacacgacgctct

SEQ ID NO: 96 - DNA, 62 bp, Artificial Sequence
  source: /note=“Description of Artificial Sequence Synthetic oligonucleotide”
aatgatacggcgaccaccgagatctacacacctagacacactctttccctacacgacgctct

SEQ ID NO: 97 - DNA, 62 bp, Artificial Sequence
  source: /note=“Description of Artificial Sequence Synthetic oligonucleotide”
aatgatacggcgaccaccgagatctacacgtggtatgacactctttccctacacgacgctct

SEQ ID NO: 98 - DNA, 62 bp, Artificial Sequence
  source: /note=“Description of Artificial Sequence Synthetic oligonucleotide”
aatgatacggcgaccaccgagatctacacgttatggcacactctttccctacacgacgctct

SEQ ID NO: 99 - DNA, 62 bp, Artificial Sequence
  source: /note=“Description of Artificial Sequence Synthetic oligonucleotide”
aatgatacggcgaccaccgagatctacacaacagcgaacactctttccctacacgacgctct

SEQ ID NO: 100 - DNA, 62 bp, Artificial Sequence
  source: /note=“Description of Artificial Sequence Synthetic oligonucleotide”
aatgatacggcgaccaccgagatctacacgtcctgttacactctttccctacacgacgctct

SEQ ID NO: 101 - DNA, 55 bp, Artificial Sequence
  source: /note=“Description of Artificial Sequence Synthetic oligonucleotide”
caagcagaagacggcatacgagatgcgaattggtgactggagttcagacgtgtgc

SEQ ID NO: 102 - DNA, 55 bp, Artificial Sequence
  source: /note=“Description of Artificial Sequence Synthetic oligonucleotide”
caagcagaagacggcatacgagataaccagaggtgactggagttcagacgtgtgc

SEQ ID NO: 103 - DNA, 55 bp, Artificial Sequence
  source: /note=“Description of Artificial Sequence Synthetic oligonucleotide”
caagcagaagacggcatacgagataatgccgagtgactggagttcagacgtgtgc

SEQ ID NO: 104 - DNA, 55 bp, Artificial Sequence
  source: /note=“Description of Artificial Sequence Synthetic oligonucleotide”
caagcagaagacggcatacgagatcgaagcttgtgactggagttcagacgtgtgc

SEQ ID NO: 105 - DNA, 55 bp, Artificial Sequence
  source: /note=“Description of Artificial Sequence Synthetic oligonucleotide”
caagcagaagacggcatacgagatggacagtagtgactggagttcagacgtgtgc

SEQ ID NO: 106 - DNA, 55 bp, Artificial Sequence
  source: /note=“Description of Artificial Sequence Synthetic oligonucleotide”
caagcagaagacggcatacgagattacgtgctgtgactggagttcagacgtgtgc

SEQ ID NO: 107 - DNA, 55 bp, Artificial Sequence
  source: /note=“Description of Artificial Sequence Synthetic oligonucleotide”
caagcagaagacggcatacgagatactgaagcgtgactggagttcagacgtgtgc

SEQ ID NO: 108 - DNA, 55 bp, Artificial Sequence
  source: /note=“Description of Artificial Sequence Synthetic oligonucleotide”
caagcagaagacggcatacgagattgcctaaggtgactggagttcagacgtgtgc

SEQ ID NO: 109 - DNA, 55 bp, Artificial Sequence
  source: /note=“Description of Artificial Sequence Synthetic oligonucleotide”
caagcagaagacggcatacgagattcgtgaaggtgactggagttcagacgtgtgc

SEQ ID NO: 110 - DNA, 55 bp, Artificial Sequence
  source: /note=“Description of Artificial Sequence Synthetic oligonucleotide”
caagcagaagacggcatacgagatagttcctggtgactggagttcagacgtgtgc

SEQ ID NO: 111 - DNA, 55 bp, Artificial Sequence
  source: /note=“Description of Artificial Sequence Synthetic oligonucleotide”
caagcagaagacggcatacgagatgtgtgtacgtgactggagttcagacgtgtgc

SEQ ID NO: 112 - DNA, 55 bp, Artificial Sequence
  source: /note=“Description of Artificial Sequence Synthetic oligonucleotide”
caagcagaagacggcatacgagatcttctccagtgactggagttcagacgtgtgc

SEQ ID NO: 113 - DNA, 55 bp, Artificial Sequence
  source: /note=“Description of Artificial Sequence Synthetic oligonucleotide”
caagcagaagacggcatacgagatactagcctgtgactggagttcagacgtgtgc

SEQ ID NO: 114 - DNA, 55 bp, Artificial Sequence
  source: /note=“Description of Artificial Sequence Synthetic oligonucleotide”
caagcagaagacggcatacgagatctctatgcgtgactggagttcagacgtgtgc

SEQ ID NO: 115 - DNA, 55 bp, Artificial Sequence
  source: /note=“Description of Artificial Sequence Synthetic oligonucleotide”
caagcagaagacggcatacgagatgcaacgttgtgactggagttcagacgtgtgc

SEQ ID NO: 116 - DNA, 55 bp, Artificial Sequence
  source: /note=“Description of Artificial Sequence Synthetic oligonucleotide”
caagcagaagacggcatacgagatcggatagtgtgactggagttcagacgtgtgc

SEQ ID NO: 117 - DNA, 55 bp, Artificial Sequence
  source: /note=“Description of Artificial Sequence Synthetic oligonucleotide”
caagcagaagacggcatacgagatcctgacaagtgactggagttcagacgtgtgc

SEQ ID NO: 118 - DNA, 55 bp, Artificial Sequence
  source: /note=“Description of Artificial Sequence Synthetic oligonucleotide”
caagcagaagacggcatacgagatcacaaggagtgactggagttcagacgtgtgc

SEQ ID NO: 119 - DNA, 55 bp, Artificial Sequence
  source: /note=“Description of Artificial Sequence Synthetic oligonucleotide”
caagcagaagacggcatacgagatgtactcctgtgactggagttcagacgtgtgc

SEQ ID NO: 120 - DNA, 55 bp, Artificial Sequence
  source: /note=“Description of Artificial Sequence Synthetic oligonucleotide”
caagcagaagacggcatacgagatagacgagagtgactggagttcagacgtgtgc

SEQ ID NO: 121 - DNA, 55 bp, Artificial Sequence
  source: /note=“Description of Artificial Sequence Synthetic oligonucleotide”
caagcagaagacggcatacgagatgaagcaaggtgactggagttcagacgtgtgc

SEQ ID NO: 122 - DNA, 55 bp, Artificial Sequence
  source: /note=“Description of Artificial Sequence Synthetic oligonucleotide”
caagcagaagacggcatacgagatctcgagttgtgactggagttcagacgtgtgc

SEQ ID NO: 123 - DNA, 55 bp, Artificial Sequence
  source: /note=“Description of Artificial Sequence Synthetic oligonucleotide”
caagcagaagacggcatacgagatctaagtcggtgactggagttcagacgtgtgc

SEQ ID NO: 124 - DNA, 55 bp, Artificial Sequence
  source: /note=“Description of Artificial Sequence Synthetic oligonucleotide”
caagcagaagacggcatacgagatgcgttagagtgactggagttcagacgtgtgc

SEQ ID NO: 125 - DNA, 55 bp, Artificial Sequence
  source: /note=“Description of Artificial Sequence Synthetic oligonucleotide”
caagcagaagacggcatacgagatggttcaacgtgactggagttcagacgtgtgc

SEQ ID NO: 126 - DNA, 55 bp, Artificial Sequence
  source: /note=“Description of Artificial Sequence Synthetic oligonucleotide”
caagcagaagacggcatacgagatacgtggttgtgactggagttcagacgtgtgc

SEQ ID NO: 127 - DNA, 55 bp, Artificial Sequence
  source: /note=“Description of Artificial Sequence Synthetic oligonucleotide”
caagcagaagacggcatacgagataccatgtcgtgactggagttcagacgtgtgc

SEQ ID NO: 128 - DNA, 55 bp, Artificial Sequence
  source: /note=“Description of Artificial Sequence Synthetic oligonucleotide”
caagcagaagacggcatacgagatgagtctcagtgactggagttcagacgtgtgc

SEQ ID NO: 129 - DNA, 55 bp, Artificial Sequence
  source: /note=“Description of Artificial Sequence Synthetic oligonucleotide”
caagcagaagacggcatacgagattgatcctcgtgactggagttcagacgtgtgc

SEQ ID NO: 130 - DNA, 55 bp, Artificial Sequence
  source: /note=“Description of Artificial Sequence Synthetic oligonucleotide”
caagcagaagacggcatacgagatcataaccggtgactggagttcagacgtgtgc

SEQ ID NO: 131 - DNA, 55 bp, Artificial Sequence
  source: /note=“Description of Artificial Sequence Synthetic oligonucleotide”
caagcagaagacggcatacgagattctactccgtgactggagttcagacgtgtgc

SEQ ID NO: 132 - DNA, 55 bp, Artificial Sequence
  source: /note=“Description of Artificial Sequence Synthetic oligonucleotide”
caagcagaagacggcatacgagattgtcgagtgtgactggagttcagacgtgtgc

SEQ ID NO: 133 - DNA, 55 bp, Artificial Sequence
  source: /note=“Description of Artificial Sequence Synthetic oligonucleotide”
caagcagaagacggcatacgagatctactctggtgactggagttcagacgtgtgc

SEQ ID NO: 134 - DNA, 55 bp, Artificial Sequence
  source: /note=“Description of Artificial Sequence Synthetic oligonucleotide”
caagcagaagacggcatacgagatacgcgaatgtgactggagttcagacgtgtgc

SEQ ID NO: 135 - DNA, 55 bp, Artificial Sequence
  source: /note=“Description of Artificial Sequence Synthetic oligonucleotide”
caagcagaagacggcatacgagataccatcgagtgactggagttcagacgtgtgc

SEQ ID NO: 136 - DNA, 55 bp, Artificial Sequence
  source: /note=“Description of Artificial Sequence Synthetic oligonucleotide”
caagcagaagacggcatacgagatgtcacttcgtgactggagttcagacgtgtgc

SEQ ID NO: 137 - DNA, 55 bp, Artificial Sequence
  source: /note=“Description of Artificial Sequence Synthetic oligonucleotide”
caagcagaagacggcatacgagatcatgagaggtgactggagttcagacgtgtgc

SEQ ID NO: 138 - DNA, 55 bp, Artificial Sequence
  source: /note=“Description of Artificial Sequence Synthetic oligonucleotide”
caagcagaagacggcatacgagatagattgcggtgactggagttcagacgtgtgc

SEQ ID NO: 139 - DNA, 55 bp, Artificial Sequence
  source: /note=“Description of Artificial Sequence Synthetic oligonucleotide”
caagcagaagacggcatacgagatgtaacggagtgactggagttcagacgtgtgc

SEQ ID NO: 140 - DNA, 55 bp, Artificial Sequence
  source: /note=“Description of Artificial Sequence Synthetic oligonucleotide”
caagcagaagacggcatacgagatcaacatcggtgactggagttcagacgtgtgc

SEQ ID NO: 141 - DNA, 55 bp, Artificial Sequence
  source: /note=“Description of Artificial Sequence Synthetic oligonucleotide”
caagcagaagacggcatacgagatggatgactgtgactggagttcagacgtgtgc

SEQ ID NO: 142 - DNA, 55 bp, Artificial Sequence
  source: /note=“Description of Artificial Sequence Synthetic oligonucleotide”
caagcagaagacggcatacgagattccgtatcgtgactggagttcagacgtgtgc

SEQ ID NO: 143 - DNA, 55 bp, Artificial Sequence
  source: /note=“Description of Artificial Sequence Synthetic oligonucleotide”
caagcagaagacggcatacgagatagtgtcgtgtgactggagttcagacgtgtgc

SEQ ID NO: 144 - DNA, 55 bp, Artificial Sequence
  source: /note=“Description of Artificial Sequence Synthetic oligonucleotide”
caagcagaagacggcatacgagattagaagccgtgactggagttcagacgtgtgc

SEQ ID NO: 145 - DNA, 55 bp, Artificial Sequence
  source: /note=“Description of Artificial Sequence Synthetic oligonucleotide”
caagcagaagacggcatacgagatgttccagagtgactggagttcagacgtgtgc

SEQ ID NO: 146 - DNA, 55 bp, Artificial Sequence
  source: /note=“Description of Artificial Sequence Synthetic oligonucleotide”
caagcagaagacggcatacgagatgttcgtcagtgactggagttcagacgtgtgc

SEQ ID NO: 147 - DNA, 55 bp, Artificial Sequence
  source: /note=“Description of Artificial Sequence Synthetic oligonucleotide”
caagcagaagacggcatacgagatcggtgaatgtgactggagttcagacgtgtgc

SEQ ID NO: 148 - DNA, 55 bp, Artificial Sequence
  source: /note=“Description of Artificial Sequence Synthetic oligonucleotide”
caagcagaagacggcatacgagattgtaacgcgtgactggagttcagacgtgtgc

SEQ ID NO: 149 - DNA, 55 bp, Artificial Sequence
  source: /note=“Description of Artificial Sequence Synthetic oligonucleotide”
caagcagaagacggcatacgagatttgccagtgtgactggagttcagacgtgtgc

SEQ ID NO: 150 - DNA, 55 bp, Artificial Sequence
  source: /note=“Description of Artificial Sequence Synthetic oligonucleotide”
caagcagaagacggcatacgagattaagctccgtgactggagttcagacgtgtgc

SEQ ID NO: 151 - DNA, 55 bp, Artificial Sequence
  source: /note=“Description of Artificial Sequence Synthetic oligonucleotide”
caagcagaagacggcatacgagatcatctcgtgtgactggagttcagacgtgtgc

SEQ ID NO: 152 - DNA, 55 bp, Artificial Sequence
  source: /note=“Description of Artificial Sequence Synthetic oligonucleotide”
caagcagaagacggcatacgagatcattgctggtgactggagttcagacgtgtgc

SEQ ID NO: 153 - DNA, 55 bp, Artificial Sequence
  source: /note=“Description of Artificial Sequence Synthetic oligonucleotide”
caagcagaagacggcatacgagatggctgatagtgactggagttcagacgtgtgc

SEQ ID NO: 154 - DNA, 55 bp, Artificial Sequence
  source: /note=“Description of Artificial Sequence Synthetic oligonucleotide”
caagcagaagacggcatacgagataggacgatgtgactggagttcagacgtgtgc

SEQ ID NO: 155 - DNA, 55 bp, Artificial Sequence
  source: /note=“Description of Artificial Sequence Synthetic oligonucleotide”
caagcagaagacggcatacgagatgaaggcatgtgactggagttcagacgtgtgc

SEQ ID NO: 156 - DNA, 55 bp, Artificial Sequence
  source: /note=“Description of Artificial Sequence Synthetic oligonucleotide”
caagcagaagacggcatacgagatgttgtggagtgactggagttcagacgtgtgc

SEQ ID NO: 157 - DNA, 55 bp, Artificial Sequence
  source: /note=“Description of Artificial Sequence Synthetic oligonucleotide”
caagcagaagacggcatacgagatatgtagccgtgactggagttcagacgtgtgc

SEQ ID NO: 158 - DNA, 55 bp, Artificial Sequence
  source: /note=“Description of Artificial Sequence Synthetic oligonucleotide”
caagcagaagacggcatacgagatttcagctcgtgactggagttcagacgtgtgc

SEQ ID NO: 159 - DNA, 55 bp, Artificial Sequence
  source: /note=“Description of Artificial Sequence Synthetic oligonucleotide”
caagcagaagacggcatacgagatccatgatggtgactggagttcagacgtgtgc

SEQ ID NO: 160 - DNA, 55 bp, Artificial Sequence
  source: /note=“Description of Artificial Sequence Synthetic oligonucleotide”
caagcagaagacggcatacgagatgccgattagtgactggagttcagacgtgtgc

SEQ ID NO: 161 - DNA, 55 bp, Artificial Sequence
  source: /note=“Description of Artificial Sequence Synthetic oligonucleotide”
caagcagaagacggcatacgagattaccaggtgtgactggagttcagacgtgtgc

SEQ ID NO: 162 - DNA, 55 bp, Artificial Sequence
  source: /note=“Description of Artificial Sequence Synthetic oligonucleotide”
caagcagaagacggcatacgagattcgtctacgtgactggagttcagacgtgtgc

SEQ ID NO: 163 - DNA, 55 bp, Artificial Sequence
  source: /note=“Description of Artificial Sequence Synthetic oligonucleotide”
caagcagaagacggcatacgagatagcatcaggtgactggagttcagacgtgtgc

SEQ ID NO: 164 - DNA, 55 bp, Artificial Sequence
  source: /note=“Description of Artificial Sequence Synthetic oligonucleotide”
caagcagaagacggcatacgagatacacttcggtgactggagttcagacgtgtgc

SEQ ID NO: 165 - DNA, 55 bp, Artificial Sequence
  source: /note=“Description of Artificial Sequence Synthetic oligonucleotide”
caagcagaagacggcatacgagatcgatgcaagtgactggagttcagacgtgtgc

SEQ ID NO: 166 - DNA, 55 bp, Artificial Sequence
  source: /note=“Description of Artificial Sequence Synthetic oligonucleotide”
caagcagaagacggcatacgagatctgtaccagtgactggagttcagacgtgtgc

SEQ ID NO: 167 - DNA, 55 bp, Artificial Sequence
  source: /note=“Description of Artificial Sequence Synthetic oligonucleotide”
caagcagaagacggcatacgagatatggtgtcgtgactggagttcagacgtgtgc

SEQ ID NO: 168 - DNA, 55 bp, Artificial Sequence
  source: /note=“Description of Artificial Sequence Synthetic oligonucleotide”
caagcagaagacggcatacgagattacggttggtgactggagttcagacgtgtgc

SEQ ID NO: 169 - DNA, 55 bp, Artificial Sequence
  source: /note=“Description of Artificial Sequence Synthetic oligonucleotide”
caagcagaagacggcatacgagatgtccataggtgactggagttcagacgtgtgc

SEQ ID NO: 170 - DNA, 55 bp, Artificial Sequence
  source: /note=“Description of Artificial Sequence Synthetic oligonucleotide”
caagcagaagacggcatacgagattactgcaggtgactggagttcagacgtgtgc

SEQ ID NO: 171 - DNA, 55 bp, Artificial Sequence
  source: /note=“Description of Artificial Sequence Synthetic oligonucleotide”
caagcagaagacggcatacgagattgtaggacgtgactggagttcagacgtgtgc

SEQ ID NO: 172 - DNA, 55 bp, Artificial Sequence
  source: /note=“Description of Artificial Sequence Synthetic oligonucleotide”
caagcagaagacggcatacgagattagccacagtgactggagttcagacgtgtgc

SEQ ID NO: 173 - DNA, 55 bp, Artificial Sequence
  source: /note=“Description of Artificial Sequence Synthetic oligonucleotide”
caagcagaagacggcatacgagattcgttcgtgtgactggagttcagacgtgtgc

SEQ ID NO: 174 - DNA, 55 bp, Artificial Sequence
  source: /note=“Description of Artificial Sequence Synthetic oligonucleotide”
caagcagaagacggcatacgagatacgaaggtgtgactggagttcagacgtgtgc

SEQ ID NO: 175 - DNA, 55 bp, Artificial Sequence
  source: /note=“Description of Artificial Sequence Synthetic oligonucleotide”
caagcagaagacggcatacgagatgccagtatgtgactggagttcagacgtgtgc

SEQ ID NO: 176 - DNA, 55 bp, Artificial Sequence
  source: /note=“Description of Artificial Sequence Synthetic oligonucleotide”
caagcagaagacggcatacgagataattcgccgtgactggagttcagacgtgtgc

SEQ ID NO: 177 - DNA, 55 bp, Artificial Sequence
  source: /note=“Description of Artificial Sequence Synthetic oligonucleotide”
caagcagaagacggcatacgagatattgctccgtgactggagttcagacgtgtgc

SEQ ID NO: 178 - DNA, 55 bp, Artificial Sequence
  source: /note=“Description of Artificial Sequence Synthetic oligonucleotide”
caagcagaagacggcatacgagatcgaatcgagtgactggagttcagacgtgtgc

SEQ ID NO: 179 - DNA, 55 bp, Artificial Sequence
  source: /note=“Description of Artificial Sequence Synthetic oligonucleotide”
caagcagaagacggcatacgagatccagaatcgtgactggagttcagacgtgtgc

SEQ ID NO: 180 - DNA, 55 bp, Artificial Sequence
  source: /note=“Description of Artificial Sequence Synthetic oligonucleotide”
caagcagaagacggcatacgagatgatccactgtgactggagttcagacgtgtgc

SEQ ID NO: 181 - DNA, 55 bp, Artificial Sequence
  source: /note=“Description of Artificial Sequence Synthetic oligonucleotide”
caagcagaagacggcatacgagatgcaatacggtgactggagttcagacgtgtgc

SEQ ID NO: 182 - DNA, 55 bp, Artificial Sequence
  source: /note=“Description of Artificial Sequence Synthetic oligonucleotide”
caagcagaagacggcatacgagattccttggagtgactggagttcagacgtgtgc

SEQ ID NO: 183 - DNA, 55 bp, Artificial Sequence
  source: /note=“Description of Artificial Sequence Synthetic oligonucleotide”
caagcagaagacggcatacgagatgattgagcgtgactggagttcagacgtgtgc

SEQ ID NO: 184 - DNA, 55 bp, Artificial Sequence
  source: /note=“Description of Artificial Sequence Synthetic oligonucleotide”
caagcagaagacggcatacgagatcagcttctgtgactggagttcagacgtgtgc

SEQ ID NO: 185 - DNA, 55 bp, Artificial Sequence
  source: /note=“Description of Artificial Sequence Synthetic oligonucleotide”
caagcagaagacggcatacgagatctcgtcatgtgactggagttcagacgtgtgc

SEQ ID NO: 186 - DNA, 55 bp, Artificial Sequence
  source: /note=“Description of Artificial Sequence Synthetic oligonucleotide”
caagcagaagacggcatacgagatataccgtcgtgactggagttcagacgtgtgc

SEQ ID NO: 187 - DNA, 55 bp, Artificial Sequence
  source: /note=“Description of Artificial Sequence Synthetic oligonucleotide”
caagcagaagacggcatacgagatgatgaaccgtgactggagttcagacgtgtgc

SEQ ID NO: 188 - DNA, 55 bp, Artificial Sequence
  source: /note=“Description of Artificial Sequence Synthetic oligonucleotide”
caagcagaagacggcatacgagatatagccaggtgactggagttcagacgtgtgc

SEQ ID NO: 189 - DNA, 55 bp, Artificial Sequence
  source: /note=“Description of Artificial Sequence Synthetic oligonucleotide”
caagcagaagacggcatacgagataggcatacgtgactggagttcagacgtgtgc

SEQ ID NO: 190 - DNA, 55 bp, Artificial Sequence
  source: /note=“Description of Artificial Sequence Synthetic oligonucleotide”
caagcagaagacggcatacgagatcctgatctgtgactggagttcagacgtgtgc

SEQ ID NO: 191 - DNA, 55 bp, Artificial Sequence
  source: /note=“Description of Artificial Sequence Synthetic oligonucleotide”
caagcagaagacggcatacgagattccagtgagtgactggagttcagacgtgtgc

SEQ ID NO: 192 - DNA, 55 bp, Artificial Sequence
  source: /note=“Description of Artificial Sequence Synthetic oligonucleotide”
caagcagaagacggcatacgagatcagatccagtgactggagttcagacgtgtgc

SEQ ID NO: 193 - DNA, 55 bp, Artificial Sequence
  source: /note=“Description of Artificial Sequence Synthetic oligonucleotide”
caagcagaagacggcatacgagatgtatggtggtgactggagttcagacgtgtgc

SEQ ID NO: 194 - DNA, 55 bp, Artificial Sequence
  source: /note=“Description of Artificial Sequence Synthetic oligonucleotide”
caagcagaagacggcatacgagatcggtattggtgactggagttcagacgtgtgc

SEQ ID NO: 195 - DNA, 55 bp, Artificial Sequence
  source: /note=“Description of Artificial Sequence Synthetic oligonucleotide”
caagcagaagacggcatacgagatagcgacaagtgactggagttcagacgtgtgc

SEQ ID NO: 196 - DNA, 55 bp, Artificial Sequence
  source: /note=“Description of Artificial Sequence Synthetic oligonucleotide”
caagcagaagacggcatacgagatttgtcctggtgactggagttcagacgtgtgc

SEQ ID NO: 197 - DNA, 66 bp, Artificial Sequence
  source: /note=“Description of Artificial Sequence Synthetic primer”
aatgatacggcgaccaccgagatctacacgttaagcgtctttccctacacgacgctcttccgatct

SEQ ID NO: 198 - DNA, 66 bp, Artificial Sequence
  source: /note=“Description of Artificial Sequence Synthetic primer”
caagcagaagacggcatacgagatgcgaattggtgactggagttcagacgtgtgctcttccgatct

SEQ ID NO: 199 - DNA, 43 bp, Artificial Sequence
  source: /note=“Description of Artificial Sequence Synthetic oligonucleotide”
tctttccctacacgacgctcttccgatctcagatctgattaca

SEQ ID NO: 200 - DNA, 48 bp, Artificial Sequence
  source: /note=“Description of Artificial Sequence Synthetic oligonucleotide”
gattacacttttgacagatcggaagagacacgtctgaactccagtcac

SEQ ID NO: 201 - DNA, 48 bp, Artificial Sequence
  source: /note=“Description of Artificial Sequence Synthetic oligonucleotide”
tgtaatcagatctgagatcggaagagcacacgtctgaactccagtcac

SEQ ID NO: 202 - DNA, 44 bp, Artificial Sequence
  source: /note=“Description of Artificial Sequence Synthetic oligonucleotide”
tctttccctacacgacgctcttccgatctgtcaaaagtgtaatc

SEQ ID NO: 203 - DNA, 49 bp, Artificial Sequence
  source: /note=“Description of Artificial Sequence Synthetic oligonucleotide”
gattacacttttgacagatcggaagagcacacgtctgaactccagtcac

SEQ ID NO: 204 - DNA, 43 bp, Artificial Sequence
  source: /note=“Description of Artificial Sequence Synthetic oligonucleotide”
tgtaatcagatctgagatcggaagagcgtcgtgtagggaaaga

SEQ ID NO: 205 - DNA, 44 bp, Artificial Sequence
  source: /note=“Description of Artificial Sequence Synthetic oligonucleotide”
gattacacttttgacagatcggaagagcgtcgtgtagggaaaga

SEQ ID NO: 206 - DNA, 66 bp, Artificial Sequence
  source: /note=“Description of Artificial Sequence Synthetic oligonucleotide”
aatgatacggcgaccaccgagatctacaccttaagcgtctttccctacacgacgctcttccgatct

SEQ ID NO: 207 - DNA, 80 bp, Artificial Sequence
  source: /note=“Description of Artificial Sequence Synthetic oligonucleotide”
aatgatacggcgaccaccgagatctacacgttaagcgtctttccctacacgacgctcttccgatctcagatctgattaca

SEQ ID NO: 208 - DNA, 80 bp, Artificial Sequence
  source: /note=“Description of Artificial Sequence Synthetic oligonucleotide”
gattacacttttacagatcggaagagcacacgtctgaactccagtcaccaattcgcatctcgtatgccgtcttctgcttg

SEQ ID NO: 209 - DNA, 80 bp, Artificial Sequence
  source: /note=“Description of Artificial Sequence Synthetic oligonucleotide”
gttcgtcttctgccgtatgctctacgcttaaccactgacctcaagtctgcacacgacaaggctagagtctagactaatgt

SEQ ID NO: 210 - DNA, 81 bp, Artificial Sequence
  source: /note=“Description of Artificial Sequence Synthetic oligonucleotide”
ctaatgtgaaaactgtctagccttctcgcagcacatccctttctgcgaattccacatctagagccaccagcggcatagtaa

SEQ ID NO: 211 - DNA, 81 bp, Artificial Sequence
  source: /note=“Description of Artificial Sequence Synthetic oligonucleotide”
gattacacttttgacagatcggaagagcacacgtctgaactccagtcaccaattcgcatctcgtatgccgtcttctgcttg

SEQ ID NO: 212 - DNA, 80 bp, Artificial Sequence; identical to SEQ ID NO: 209
  source: /note=“Description of Artificial Sequence Synthetic oligonucleotide”

SEQ ID NO: 213 - DNA, 80 bp, Artificial Sequence
  source: /note=“Description of Artificial Sequence Synthetic oligonucleotide”
ttactatgccgctggtggctctagatgtgcaattcgcagaaagggatgtgctgcgagaaggctagagtctagactaatgt

SEQ ID NO: 214 - DNA, 80 bp, Artificial Sequence; identical to SEQ ID NO: 213
  source: /note=“Description of Artificial Sequence Synthetic oligonucleotide”

SEQ ID NO: 215 - DNA, 81 bp, Artificial Sequence; identical to SEQ ID NO: 211
  source: /note=“Description of Artificial Sequence Synthetic oligonucleotide”

SEQ ID NO: 216 - DNA, 81 bp, Artificial Sequence
  source: /note=“Description of Artificial Sequence Synthetic oligonucleotide”
gattacacttttgacagatcggaagagcgtcgtgtagggaaagacgcttaaggtgtagatctcggtggtcgccgtatcatt

SEQ ID NO: 217 - DNA, 80 bp, Artificial Sequence; identical to SEQ ID NO: 209
  source: /note=“Description of Artificial Sequence Synthetic oligonucleotide”

(19) United States
(12) Patent Application Publication
(10) Pub. No.: US 2020/0109397 A1
(43) Pub. Date: Apr. 9, 2020

(54) Modular Nucleic Acid Adapters

(71) Applicant: Roche Sequencing Solutions, Inc., Pleasanton, CA (US)

(72) Inventors: Daniel Klass, Livermore, CA (US); Alexander Lovejoy, Newark, CA (US); Seyed Hamid Mirebrahim, Dublin, CA (US); Amrita Pati, San Ramon, CA (US)

(21) Appl. No.: 16/721,533

(22) Filed: Dec. 19, 2019

Related U.S. Application Data
(63) Continuation of application No. PCT/EP2018/067246, filed on Jun. 27, 2018.
(60) Provisional application No. 62/525,595, filed on Jun. 27, 2017.

(51) Int. Cl. C12N 15/10

(52) U.S. Cl. CPC C12N 15/1093

(57) ABSTRACT

The present disclosure provides a kit for preparing a library of nucleic acids. The kit includes first and second oligonucleotide, each having a tail sequence, a common sequence, and at least one of a unique identifier sequence, and a variable length punctuation mark. The kit further includes a first primer having a first sample identifier sequence and a first priming sequence at a 3′ end of the first primer. The first priming sequence includes the tail sequence of the first oligonucleotide. The kit further includes a second primer having a second sample identifier sequence and a second priming sequence at a 3′ end of the second primer. The second priming sequence is complimentary to the second tail sequence of the second oligonucleotide.

### CROSS-REFERENCE TO RELATED APPLICATIONS

The present application claims the benefit of priority under 35 U.S.C. § 119(a) of International Application No. PCT/EP2018/067246, filed Jun. 27, 2018, which claims priority to U.S. Application Ser. No. 62/525,595, filed Jun. 27, 2017. The disclosures of each of these applications are incorporated herein by reference in their entireties.

### BACKGROUND

The disclosure relates, in general, to sample preparation for next generation sequencing of nucleic acids and, more particularly, to a system and method for the isolation and qualification of nucleic acids.

Forked nucleic acid adapters (also known as Y-adapters) for use with next generation sequencing (NGS) platforms (e.g., ILLUMINA sequencing-by-synthesis platforms) can include features such sample identifiers (SID) and unique identifiers (UID) that enable sample multiplexing, molecular counting, and the like. Accordingly, forked adapters can enable efficient NGS library preparation via adapter ligation methods, maximizing the number of molecules that can be sequenced in a paired-end fashion, while allowing correct counting of molecules and error reduction with UIDs. However, there are a number of challenges that may arise when producing and using adapter such as these.

In one aspect, the cost of oligo manufacturing is high. For an adapter design with 16 unique UIDs, in order to create adapters with 16 different single-stranded SIDs, 274 different oligo sequences must be produced. However, only a small number of oligo manufacturers are able to produce such a large number of different oligos at a high enough purity in a large enough scale to satisfy these specifications.

In another aspect, addition of PhiX to the final sequencing libraries (which can comprise 10-15% of the final concentration of the input molecules in an NGS experiment) effectively reduces the number of sequencing reads available for DNA molecules from the sample. PhiX DNA is often used as a spike-in control during library preparation as a quality control for NGS experiments or to add complexity in the case of less complex DNA samples. For example, PhiX may be used if 100% of the bases at positions 3 and 4 in the library sequences are G and T as PhiX increases the complexity at these positions, allowing the ILLUMINA sequencer to properly differentiate clusters and phase the molecules.

In yet another aspect, with 16 2-base UIDs (i.e., UIDs having a length of 2 nucleotides), any error in the UID results in a different acceptable UID. This could lead to over counting of molecules, and less efficient error reduction than UIDs that can be better differentiated.

In a further aspect, a known phenomenon that is often observed in NGS experiments involves the SID for a molecule from one sample attaching or otherwise associating with molecule from another sample. This can result in molecules being assigned to the incorrect sample. If the adapter scheme only contains an SID on one side of the adapter, and the SID is not directly attached to the molecule of interest, this crossover effect can perturb variant calling, thereby resulting in incorrect variant calls. Taken together with the other aforementioned challenges, it is clear that there is room for improvement of nucleic acid adapters for NGS experiments.

Accordingly, there is a need for new designs for nucleic acid adapters that enable lower manufacturing costs and greater efficiency and accuracy in NGS experiments.

### SUMMARY

The present invention overcomes the aforementioned drawbacks by providing a kits and methods including modular nucleic acid adapters as described by the following enumerated list:

1. A kit for preparing a library of nucleic acids having adapter sequences for sequencing, the kit comprising:

a first oligonucleotide having a first tail sequence, a first common sequence, and at least one of i) a first unique identifier sequence, and ii) a first variable length punctuation mark;

a second oligonucleotide having a second tail sequence, a second common sequence complimentary to the first common sequence, and at least one of i) a second unique identifier sequence complimentary to the first unique identifier sequence, and ii) a second variable length punctuation mark complimentary to the first variable length punctuation mark;

a first primer having a first sample identifier sequence and a first priming sequence at a 3′ end of the first primer, the first priming sequence including the first tail sequence of the first oligonucleotide; and

a second primer having a second sample identifier sequence and a second priming sequence at a 3′ end of the second primer, the second priming sequence being complimentary to the second tail sequence of the second oligonucleotide.

2. The kit of 1, wherein the first sample identifier sequence and the second sample identifier sequence have a one-to-one mapping.

3. The kit of 2, wherein the first variable length punctuation mark has a length of 2-4 nucleotides.

4. The kit of 2, where the first variable length punctuation mark includes at least one of a G and a C nucleotide.

5. The kit of 1, wherein the first unique identifier sequence has a length of at least 5 nucleotides.

6. The kit of 5, wherein the first unique identifier sequence has a pairwise edit distance of at least 3.

7. A kit for preparing a library of nucleic acids having adapter sequences for sequencing, the kit comprising:

a plurality of oligonucleotide pairs, each of the oligonucleotide pairs including:
a first oligonucleotide having a first tail sequence, a first common sequence, and at least one of i) a first unique identifier sequence, and ii) a first variable length punctuation mark, and
a second oligonucleotide having a second tail sequence, a second common sequence complimentary to the first common sequence, and at least one of i) a second unique identifier sequence complimentary to the first unique identifier sequence, and ii) a second variable length punctuation mark complimentary to the first variable length punctuation mark,
a first primer having a first sample identifier sequence and a first priming sequence at a 3′ end of the first primer, the first priming sequence including the first tail sequence of the first oligonucleotide; and
a second primer having a second sample identifier sequence and a second priming sequence at a 3′ end of the second primer, the second priming sequence being complimentary to the second tail sequence of the second oligonucleotide.

8. The kit of 7, wherein each of the first unique identifier sequences of each of the plurality of oligonucleotide pairs is different.

9. The kit of 7, wherein each of the first tail sequences of each of the plurality of oligonucleotide pairs is the same.

10. The kit of 7, wherein each of the second tail sequences of each of the plurality of oligonucleotide pairs is the same.

11. The kit of 7, wherein each of the plurality of oligonucleotide pairs are annealed to form a forked adapter.

12. The kit of 7, wherein the first sample identifier sequence and the second sample identifier sequence have a one-to-one mapping.

13. The kit of 12, wherein each of the first variable length punctuation marks has a length of 2-4 nucleotides.

14. The kit of 12, where each of the first variable length punctuation marks includes at least one of a G and a C nucleotide.

15. The kit of 7, wherein each of the first unique identifier sequences has a length of at least 5 nucleotides.

16. The kit of 15, wherein each of the first unique identifier sequences has a pairwise edit distance of at least 3.

17. A method of preparing a library of nucleic acid molecules, the method comprising:

attaching one of a plurality of oligonucleotide adapters to each end of a target nucleic acid to provide an adapter-target-adapter construct, each of the plurality of oligonucleotide adapters having:
a first oligonucleotide having a first tail sequence, a first common sequence, and at least one of i) a first unique identifier sequence, and ii) a first variable length punctuation mark, and
a second oligonucleotide having a second tail sequence, a second common sequence complimentary to the first common sequence, and at least one of i) a second unique identifier sequence complimentary to the first unique identifier sequence, and ii) a second variable length punctuation mark complimentary to the first variable length punctuation mark;

annealing a first primer to the adapter-target-adapter construct, the first primer having a first sample identifier sequence and a first priming sequence at a 3′ end of the first primer, the first priming sequence including the first tail sequence of the first oligonucleotide; and

extending each of the first primer and the second primer to form extension products complementary to each strand of the adapter-target-adapter constructs.

18. The method of 17, wherein each of the first unique identifier sequences of each of the plurality of oligonucleotide adapters is different.

19. The method of 17, wherein each of the first tail sequences of each of the plurality of oligonucleotide adapters is the same.

20. The method of 17, wherein each of the second tail sequences of each of the plurality of oligonucleotide adapters is the same.

21. The method of 17, wherein the first sample identifier sequence and the second sample identifier sequence have a one-to-one mapping.

22. The method of 21, wherein each of the first variable length punctuation marks has a length of 2-4 nucleotides.

23. The method of 21, where each of the first variable length punctuation marks includes at least one of a G and a C nucleotide.

24. The method of 17, wherein each of the first unique identifier sequences has a length of at least 5 nucleotides.

25. The method of 24, wherein each of the first unique identifier sequences has a pairwise edit distance of at least 3.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a schematic diagram depicting an embodiment of the components of a modular nucleic acid adapter according to the present disclosure.

FIG. 2A is a schematic illustration of a method for preparing a library of nucleic acids with the modular nucleic acid adapters according the present disclosure. In a first portion of the method, a scheme is illustrated for assembling a pool of adapter oligos, including the design of adapter oligos having predetermined molecular barcodes (UIDs) and forward and reverse primers having SIDs for amplification of the adaptor oligos following ligation to sample nucleic acid library fragments. In the present example, each sample nucleic acid fragment is ligated at each end to one of the 16 different annealed adapters (each of the annealed adapters having one of 16 predetermined molecular barcodes or UIDs). Following ligation, each nucleic acid fragment in the sample is associated with one of 256 different possible pairs of molecular barcode sequences. FIG. 2A discloses SEQ ID NOS 3, 4, 3, 4, 197 and 198, respectively, in the order of their appearance.

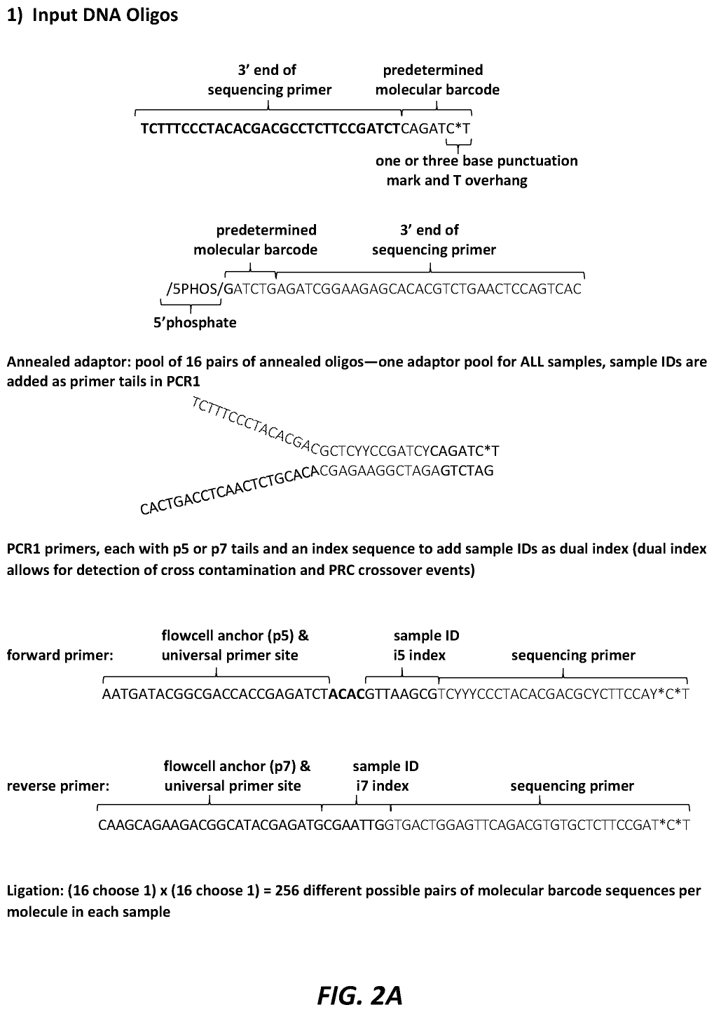

FIG. 2B is a continuation of the schematic illustration of the method of FIG. 2A. Following ligation of the annealed adapters to the target DNA molecules in the nucleic acid sample, the primers having SIDs illustrated in FIG. 2A are used in first and second rounds of a polymerase chain reaction (PCR) experiment to incorporate SIDs and NGS platform specific sequences (e.g., p5 and p7 sequences for ILLUMINA sequencers). FIG. 2B discloses SEQ ID NOS 199-203, 198, 197 and 204-206, respectively, in the order of their appearance.

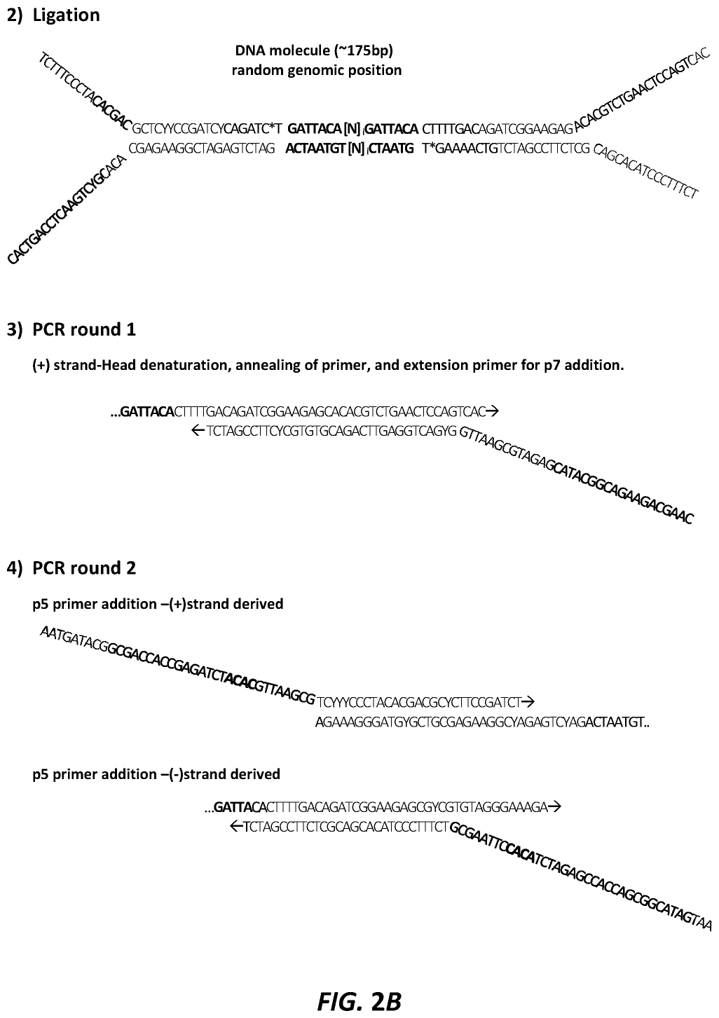

FIG. 2C is a continuation of the schematic illustration of the method of FIGS. 2A and 2B. Following PCR amplification, the illustrated PCR products are subjected to sequencing. In the present example, the relevant priming sites for sequencing on an ILLUMINA platform (e.g., ILLUMINA HISEQ series) are indicated with underlining for each of the PCR products. FIG. 2C discloses SEQ ID NOS 207-217, respectively, in the order of their appearance.

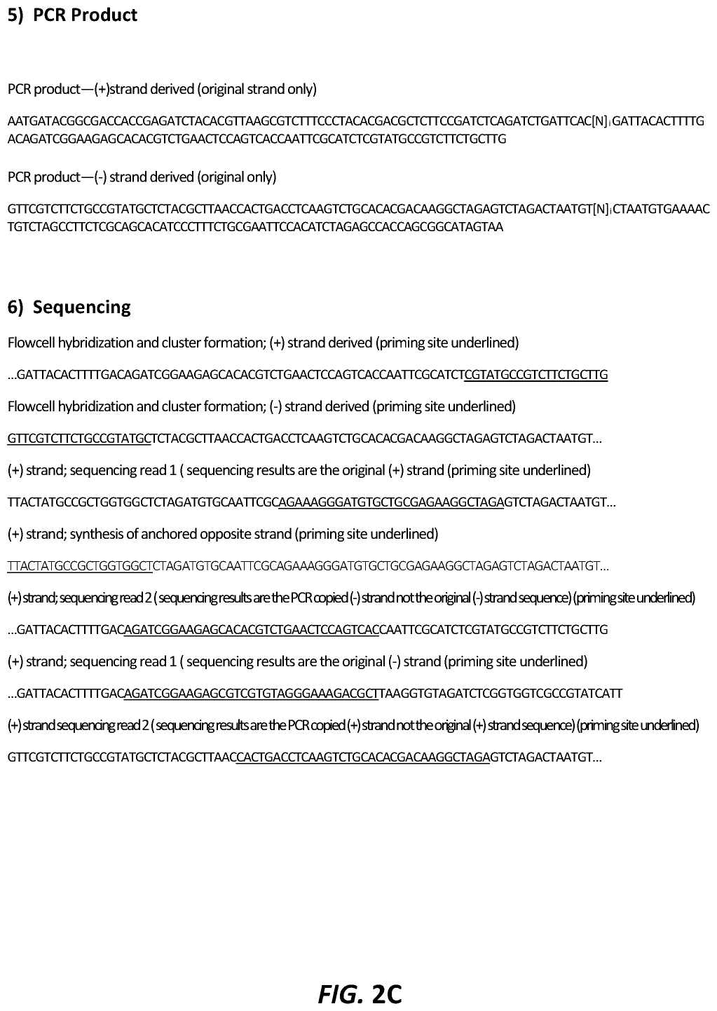

### DETAILED DESCRIPTION

### I. Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

Approximately: As used herein, the term “approximately” or “about”, as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level, and/or form of one is correlated with that of the other. For example, a particular entity (e.g., polypeptide, genetic signature, metabolite, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biological Sample: As used herein, the term “biological sample” typically refers to a sample obtained or derived from a biological source (e.g., a tissue or organism or cell culture) of interest, as described herein. In some embodiments, a source of interest comprises or consists of an organism, such as an animal or human. In some embodiments, a biological sample is comprises or consists of biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; other body fluids, secretions, and/or excretions; and/or cells therefrom, etc. In some embodiments, a biological sample is comprises or consists of cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Comprising: A composition or method described herein as “comprising” one or more named elements or steps is open-ended, meaning that the named elements or steps are essential, but other elements or steps may be added within the scope of the composition or method. It is to be understood that composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Designed: As used herein, the term “designed” refers to an agent (i) whose structure is or was selected by the hand of man; (ii) that is produced by a process requiring the hand of man; and/or (iii) that is distinct from natural substances and other known agents.

Determine: Those of ordinary skill in the art, reading the present specification, will appreciate that “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Sample: As used herein, the term “sample” refers to a substance that is or contains a composition of interest for qualitative and or quantitative assessment. In some embodiments, a sample is a biological sample (i.e., comes from a living thing (e.g., cell or organism). In some embodiments, a sample is from a geological, aquatic, astronomical, or agricultural source. In some embodiments, a source of interest comprises or consists of an organism, such as an animal or human. In some embodiments, a sample for forensic analysis is or comprises biological tissue, biological fluid, organic or non-organic matter such as, e.g., clothing, dirt, plastic, water. In some embodiments, an agricultural sample, comprises or consists of organic matter such as leaves, petals, bark, wood, seeds, plants, fruit, etc.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Synthetic: As used herein, the word “synthetic” means produced by the hand of man, and therefore in a form that does not exist in nature, either because it has a structure that does not exist in nature, or because it is either associated with one or more other components, with which it is not associated in nature, or not associated with one or more other components with which it is associated in nature.

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc.) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to another in linear or three-dimensional space. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. In some embodiments, a variant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide. In many embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 additions or deletions, and often has no additions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, a variant may also have one or more functional defects and/or may otherwise be considered a “mutant”. In some embodiments, the parent or reference polypeptide is one found in nature. As will be understood by those of ordinary skill in the art, a plurality of variants of a particular polypeptide of interest may commonly be found in nature, particularly when the polypeptide of interest is an infectious agent polypeptide.

### II. Detailed Description of Certain Embodiments

As also discussed above, in various situations it may be useful to provide adapters for nucleic acid library preparation for NGS or the like. However, current adapter designs have several drawbacks with respect to cost of manufacture, efficiency of sequencing and accuracy of downstream base-calling, sample identification, and the like.

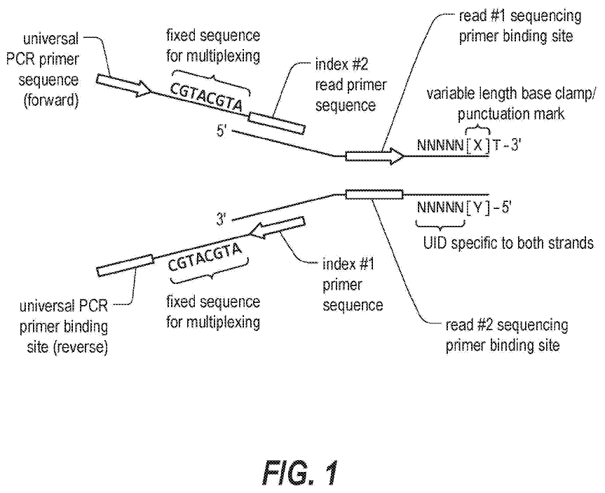

These and other challenges may be overcome with a modular nucleic acid adapter according to the present disclosure. In one aspect, the disclosed adapters may be implemented to overcome the aforementioned challenges using a scheme whereby UIDs and SIDs are distributed onto two separate sets of oligos (FIG. 1). Accordingly, in one embodiment, a pool of forked adapter is prepared with each adapter having a UID selected from a set of two or more different UID sequences. Following ligation of the UID-containing forked adapters to target nucleic acids, the resulting ligation products are amplified with primers including SIDs, and optionally other sequence information such as NGS platform specific sequences. The resulting amplification products include both a pair of UIDs from the initial adapter ligation step and an SID (or pair of SIDs) from the amplification step. Notably, variations of the aforementioned modular design are also within the scope of the present disclosure. For example, the location of the UIDs and SIDs can be swapped. That is, the UIDs on the forked adapters can be substituted for SID and the SIDs included in the amplification primers can be substituted for UIDs. As a result, the SIDs are incorporated by ligation and the UIDs are incorporated through PCR amplification. Yet other variations of the disclosed modular nucleic acid adapters will become apparent from the following disclosure.

One advantage of the disclosed modular nucleic acid adapter design is that instead of each adapter having its own SID, then being amplified by a universal PCR primer pair, the adapter is universal (e.g., adapters with 16 different UIDs are pooled into one adapter tube), and the PCR primers contain the SIDs. In this design, the UIDs and SIDs are decoupled, allowing a reduction in the number of necessary oligos to be produced. For an adapter design with 16 different UIDs and 16 SIDs, 64 different oligos are necessary, instead of 274. In addition, these oligos are shorter than those in the previous design, which also reduces oligo synthesis costs, and may increase efficiency of ligation (and therefore assay efficiency) as well. In one aspect, the set of different UIDs includes 2, 4, 8, 16, 32, 64, 128, or more different UID sequences. In another aspect, the set of different SIDs includes 2, 4, 8, 16, 32, 64, 128, or more different SID sequences. Notably, the number of UIDs and SIDs selected will depend on the nature of the experiment including the desired number of samples for multiplexing, the capacity of the NGS platform (i.e., the sequencing instrument), the complexity of the nucleic acid sample to be analyzed, and the like.

In another aspect of the disclosed modular nucleic acid adapter design, instead of having a consistent 2-base punctuation mark of GT at the end of every adapter, the punctuation mark is synthesized with a variable length. The use of a variable length punctuation mark (FIG. 1) ensure adequate complexity at each position within the read, so a PhiX spike-in or other like control or complexity enhancing material is not needed. In the one embodiment, the punctuation mark is varied between 2- and 4-bases. In this implementation, the last base before the T-overhang is selected from a C nucleotide or a G nucleotide, thereby allowing a stronger hydrogen bond (i.e., a “G-C clamp”), which may show improved ligation efficiency. In another embodiment, the terminal base of the punctuation mark is selected from any of any nucleotide. In one aspect, the punctuation marks can be designed such that no position in the sequencing read ever has greater than a selected percentage (e.g., 62.5%) of any base at the position, removing the need for addition of PhiX or another like agent when using the disclosed adapters. A list of punctuation marks and the breakdown of base % at each position is shown in Tables 1 and 2.

TABLE 1
i5 punctuation marks (with T overhang)
C
G
AAG
TCC
C
G
AGG
TAC
C
G
TCG
AGC
C
G
TAG
ACC

TABLE 2
% Each base by position in the punctuation mark*

| Base | Position 1 | Position 2 |
|---|---|---|
| A | 25% | 18.75% |
| C | 25% | 18.75% |
| G | 25% | 12.50% |
| T | 25% | 50% |

*Assuming a nucleic acid sample having 25% representation of each base at each position

In another aspect of the present disclosure, UIDs can be designed such that, if one or multiple errors occur in the UID, the UID does not result in the same sequence as another UID in the selected pool of UID sequences. In this way, UIDs with one or multiple errors can be corrected or removed from further analysis. In the attached implementation, instead of UIDs with a length of 2 nucleotides, a UID with a length of 5 nucleotides with a pairwise edit distance of at least 3 are used. As defined herein, pairwise edit distance is a measure of the similarity between two strings of characters (e.g., nucleotide sequences) as determined by counting the minimum number of operations required to transform one string into the other. As used in the examples of the present disclosure, pairwise edit distance is determined according the Levenshtein distance, in which operations are limited to deletions, insertions, and substitutions; however, pairwise edit distance may be calculated using other approaches as will be appreciated by one of ordinary skill in the art. With a pairwise edit distance of 3, UIDs having a single error can always be identified correctly. This allows for up to 25 different UIDs (see, e.g., Faircloth, et al. 2012. PLoS ONE 7(8): e42543). In the attached implementation (Table 3), 16 UIDs are used. Different length UIDs can also be used (e.g., designs with UIDs as short as 2 and as long as 10 bases in length). With 2 base UIDs and the use of a variable punctuation mark as described herein, UIDs+punctuation marks with a pairwise hamming distance of 2 can be generated-in this implementation (Table 4), one substitution error in the UID will never result in a UID+punctuation mark sequence that is identical to another UID+punctuation mark in the set. As defined herein, hamming distance is the edit distance between two strings where the only allowed operation is a substitution. Two additional UID schemes are shown in Tables 5 and 6 below.

TABLE 3
(scheme 1)

| UID | rc UID | i5 punc | i7 punc |
|---|---|---|---|
| CAGAT | ATCTG | C | G |
| GCTGA | TCAGC | G | C |
| GTCAA | TTGAC | AAG | CTT |
| GACGT | ACGTC | TCC | GGA |
| AGGTG | CACCT | C | G |
| GTACC | GGTAC | G | C |
| CGCTT | AAGCG | AGG | CCT |
| AACCG | CGGTT | TAC | GTA |
| ACTTC | GAAGT | C | G |
| TCGGT | ACCGA | G | C |
| CCTAG | CTAGG | TCG | CGA |
| CATCC | GGATG | AGC | GCT |
| TCATG | CATGA | C | G |
| ATGCA | TGCAT | G | C |
| GGAAT | ATTCC | TAG | CTA |
| TTGAC | GTCAA | ACC | GGT |

TABLE 4
(scheme 4)

| UID | rc UID | i5 punc | i7 punc |
|---|---|---|---|
| AA | TT | TCC | GGA |
| AC | GT | C | G |
| AG | CT | AAG | CTT |
| AT | AT | G | C |
| CA | TG | G | C |
| CC | GG | AGG | CCT |
| CG | CG | C | G |
| CT | AG | TAC | GTA |
| GA | TC | AGC | GCT |
| GC | GC | G | C |
| GG | CC | TCG | CGA |
| GT | AC | C | G |
| TA | TA | C | G |
| TC | GA | TAG | CTA |
| TG | CA | G | C |
| TT | AA | ACC | GGT |

TABLE 5
(scheme 2)

| UID | rc UID | i5 punc | i7 punc |
|---|---|---|---|
| AA | TT | C | G |
| AC | GT | G | C |
| AG | CT | AAG | CTT |
| AT | AT | TCC | GGA |
| CA | TG | C | G |
| CC | GG | G | C |
| CG | CG | AGG | CCT |
| CT | AG | TAC | GTA |
| GA | TC | C | G |
| GC | GC | G | C |
| GG | CC | TCG | CGA |
| GT | AC | AGC | GCT |
| TA | TA | C | G |
| TC | GA | G | C |
| TG | CA | TAG | CTA |
| TT | AA | ACC | GGT |

TABLE 6
(scheme 3)

| UID | rc UID | i5 punc | i7 punc |
|---|---|---|---|
| AA | TT | C | G |
| AC | GT | G | C |
| AG | CT | C | G |
| AT | AT | G | C |
| CA | TG | C | G |
| CC | GG | G | C |
| CG | CG | C | G |
| CT | AG | G | C |
| GA | TC | C | G |
| GC | GC | G | C |
| GG | CC | C | G |
| GT | AC | G | C |
| TA | TA | C | G |
| TC | GA | G | C |
| TG | CA | C | G |
| TT | AA | G | C |

Referring to the adapter schemes illustrated in Tables 3-6, the UID and punctuation mark can be combined with any suitable adapter sequence. For example, the ILLUMINA i5 and i7 adapter sequences are TCTTTCCCTACACGACGCTCTTCCGATCT (SEQ ID NO:1) and AGATCGGAAGAGCACACGTCTGAACTCCAGTCAC (SEQ ID NO: 2), respectively. The UID sequence (UID) CAGAT and the i5 punctuation mark (i5 punc) C in the first row of Table 3 can be combined with the ILLUMINA i5 adapter sequence to provide the oligo sequence TCTTTCCCTACACGACGCTCTTCCGATCTCAGATC*T (SEQ ID NO: 3), where the asterisk (*) indicates a phosphorothioate bond. Similarly, the reverse complement of the UID (rc UID) ATCTG and the i7 punctuation mark (i7 punc) G (the reverse complement of the i5 punctuation mark C) can be combined with the ILLUMINA i7 adapter sequence to provide the oligo sequence GATCTGAGATCGGAAGAGCACACGTCTGAACTCCAGTCAC (SEQ ID NO: 4), where the sequence includes a 5′-phosphate group. Each of Tables 3-6 lists a set of 16 different UID/punctuation mark combinations that can be used to prepare a set of 16 oligonucleotide pairs.

For preparation of adapters, each of the oligonucleotide pairs is synthesized, purified, and annealed to provide a homogenous population of annealed adapters. Then the 16 different pools of annealed adapters are combined to make one adapter pool with 16 different UIDs. It will be appreciated that pools of adapters with more or less than 16 different UIDs can also be prepared using the described approach.

In another aspect of the present disclosure, instead of an SID on only one sequencing read, an SID can be incorporated into one or both PCR primers for amplification of products resulting from ligation of target nucleic acids with annealed adapters having different UIDs. By using primers having SIDs incorporated therein, both index reads will resulting from sequencing will provide SIDs. Within one primer pair, the SIDs can be designed to have a one-to-one mapping such that when an SID from one index read is known, the SID from the other read (from the paired end) is predictable. This one-to-one mapping of SIDs enables removal of reads in an SID when a molecule from one sample associated with a first SID attaches to a molecule from another sample associated with a second SID. In the implementation shown in Tables 7 and 8, the SIDs are the reverse of each other. One sequence is considered the ‘reverse’ of another sequence when the two sequences share the same sequence of nucleotides in the reverse order. For example, if a first SID has the sequence AACT, a second SID having the sequence TCAA would be the reverse of the first SID. Notably, the reverse of a sequence is different from the reverse complement of a sequence. The SIDs have a minimum pairwise edit distance of 3, so with up to 1 error, an SID can always be properly associated with the correct SID sequence. Example SIDs useful with the present disclosure are described by Faircloth and coworkers (Faircloth, et al. 2012. PLoS ONE 7(8): e42543). While the sequences in Tables 7 and 8 include 96 SID pairs, it will be appreciated that yet other sequences, combinations, and numbers of SIDs can be used in the context of the present disclosure.

TABLE 7

| Pair | Forward Primer (SEQ. ID. NOs: 5-100) |
|---|---|
| 1 | AATGATACGGCGACCACCGAGATCTACACGTTAAGCGACACTC |
| | TTTCCCTACACGACGCTCT |
| 2 | AATGATACGGCGACCACCGAGATCTACACGAGACCAAACACTC |
| | TTTCCCTACACGACGCTCT |
| 3 | AATGATACGGCGACCACCGAGATCTACACAGCCGTAAACACTC |
| | TTTCCCTACACGACGCTCT |
| 4 | AATGATACGGCGACCACCGAGATCTACACTTCGAAGCACACTC |
| | TTTCCCTACACGACGCTCT |
| 5 | AATGATACGGCGACCACCGAGATCTACACATGACAGGACACTC |
| | TTTCCCTACACGACGCTCT |
| 6 | AATGATACGGCGACCACCGAGATCTACACTCGTGCATACACTC |
| | TTTCCCTACACGACGCTCT |
| 7 | AATGATACGGCGACCACCGAGATCTACACCGAAGTCAACACTC |
| | TTTCCCTACACGACGCTCT |
| 8 | AATGATACGGCGACCACCGAGATCTACACGAATCCGTACACTC |
| | TTTCCCTACACGACGCTCT |
| 9 | AATGATACGGCGACCACCGAGATCTACACGAAGTGCTACACTC |
| | TTTCCCTACACGACGCTCT |
| 10 | AATGATACGGCGACCACCGAGATCTACACGTCCTTGAACACTC |
| | TTTCCCTACACGACGCTCT |
| 11 | AATGATACGGCGACCACCGAGATCTACACCATGTGTGACACTC |
| | TTTCCCTACACGACGCTCT |
| 12 | AATGATACGGCGACCACCGAGATCTACACACCTCTTCACACTC |
| | TTTCCCTACACGACGCTCT |
| 13 | AATGATACGGCGACCACCGAGATCTACACTCCGATCAACACTC |
| | TTTCCCTACACGACGCTCT |
| 14 | AATGATACGGCGACCACCGAGATCTACACCGTATCTCACACTC |
| | TTTCCCTACACGACGCTCT |
| 15 | AATGATACGGCGACCACCGAGATCTACACTTGCAACGACACTC |
| | TTTCCCTACACGACGCTCT |
| 16 | AATGATACGGCGACCACCGAGATCTACACTGATAGGCACACTC |
| | TTTCCCTACACGACGCTCT |
| 17 | AATGATACGGCGACCACCGAGATCTACACAACAGTCCACACTC |
| | TTTCCCTACACGACGCTCT |
| 18 | AATGATACGGCGACCACCGAGATCTACACAGGAACACACACTC |
| | TTTCCCTACACGACGCTCT |
| 19 | AATGATACGGCGACCACCGAGATCTACACTCCTCATGACACTC |
| | TTTCCCTACACGACGCTCT |
| 20 | AATGATACGGCGACCACCGAGATCTACACAGAGCAGAACACTC |
| | TTTCCCTACACGACGCTCT |
| 21 | AATGATACGGCGACCACCGAGATCTACACGAACGAAGACACTC |
| | TTTCCCTACACGACGCTCT |
| 22 | AATGATACGGCGACCACCGAGATCTACACTTGAGCTCACACTC |
| | TTTCCCTACACGACGCTCT |
| 23 | AATGATACGGCGACCACCGAGATCTACACGCTGAATCACACTC |
| | TTTCCCTACACGACGCTCT |
| 24 | AATGATACGGCGACCACCGAGATCTACACAGATTGCGACACTC |
| | TTTCCCTACACGACGCTCT |
| 25 | AATGATACGGCGACCACCGAGATCTACACCAACTTGGACACTC |
| | TTTCCCTACACGACGCTCT |
| 26 | AATGATACGGCGACCACCGAGATCTACACTTGGTGCAACACTC |
| | TTTCCCTACACGACGCTCT |
| 27 | AATGATACGGCGACCACCGAGATCTACACCTGTACCAACACTC |
| | TTTCCCTACACGACGCTCT |
| 28 | AATGATACGGCGACCACCGAGATCTACACACTCTGAGACACTC |
| | TTTCCCTACACGACGCTCT |
| 29 | AATGATACGGCGACCACCGAGATCTACACCTCCTAGTACACTC |
| | TTTCCCTACACGACGCTCT |
| 30 | AATGATACGGCGACCACCGAGATCTACACGCCAATACACACTC |
| | TTTCCCTACACGACGCTCT |
| 31 | AATGATACGGCGACCACCGAGATCTACACCCTCATCTACACTC |
| | TTTCCCTACACGACGCTCT |
| 32 | AATGATACGGCGACCACCGAGATCTACACTGAGCTGTACACTC |
| | TTTCCCTACACGACGCTCT |
| 33 | AATGATACGGCGACCACCGAGATCTACACGTCTCATCACACTC |
| | TTTCCCTACACGACGCTCT |
| 34 | AATGATACGGCGACCACCGAGATCTACACTAAGCGCAACACTC |
| | TTTCCCTACACGACGCTCT |
| 35 | AATGATACGGCGACCACCGAGATCTACACAGCTACCAACACTC |
| | TTTCCCTACACGACGCTCT |
| 36 | AATGATACGGCGACCACCGAGATCTACACCTTCACTGACACTC |
| | TTTCCCTACACGACGCTCT |
| 37 | AATGATACGGCGACCACCGAGATCTACACGAGAGTACACACTC |
| | TTTCCCTACACGACGCTCT |
| 38 | AATGATACGGCGACCACCGAGATCTACACGCGTTAGAACACTC |
| | TTTCCCTACACGACGCTCT |
| 39 | AATGATACGGCGACCACCGAGATCTACACAGGCAATGACACTC |
| | TTTCCCTACACGACGCTCT |
| 40 | AATGATACGGCGACCACCGAGATCTACACGCTACAACACACTC |
| | TTTCCCTACACGACGCTCT |
| 41 | AATGATACGGCGACCACCGAGATCTACACTCAGTAGGACACTC |
| | TTTCCCTACACGACGCTCT |
| 42 | AATGATACGGCGACCACCGAGATCTACACCTATGCCTACACTC |
| | TTTCCCTACACGACGCTCT |
| 43 | AATGATACGGCGACCACCGAGATCTACACTGCTGTGAACACTC |
| | TTTCCCTACACGACGCTCT |
| 44 | AATGATACGGCGACCACCGAGATCTACACCCGAAGATACACTC |
| | TTTCCCTACACGACGCTCT |
| 45 | AATGATACGGCGACCACCGAGATCTACACAGACCTTGACACTC |
| | TTTCCCTACACGACGCTCT |
| 46 | AATGATACGGCGACCACCGAGATCTACACACTGCTTGACACTC |
| | TTTCCCTACACGACGCTCT |
| 47 | AATGATACGGCGACCACCGAGATCTACACTAAGTGGCACACTC |
| | TTTCCCTACACGACGCTCT |
| 48 | AATGATACGGCGACCACCGAGATCTACACCGCAATGTACACTC |
| | TTTCCCTACACGACGCTCT |
| 49 | AATGATACGGCGACCACCGAGATCTACACTGACCGTTACACTC |
| | TTTCCCTACACGACGCTCT |
| 50 | AATGATACGGCGACCACCGAGATCTACACCCTCGAATACACTC |
| | TTTCCCTACACGACGCTCT |
| 51 | AATGATACGGCGACCACCGAGATCTACACTGCTCTACACACTC |
| | TTTCCCTACACGACGCTCT |
| 52 | AATGATACGGCGACCACCGAGATCTACACGTCGTTACACACTC |
| | TTTCCCTACACGACGCTCT |
| 53 | AATGATACGGCGACCACCGAGATCTACACATAGTCGGACACTC |
| | TTTCCCTACACGACGCTCT |
| 54 | AATGATACGGCGACCACCGAGATCTACACTAGCAGGAACACTC |
| | TTTCCCTACACGACGCTCT |
| 55 | AATGATACGGCGACCACCGAGATCTACACTACGGAAGACACTC |
| | TTTCCCTACACGACGCTCT |
| 56 | AATGATACGGCGACCACCGAGATCTACACAGGTGTTGACACTC |
| | TTTCCCTACACGACGCTCT |
| 57 | AATGATACGGCGACCACCGAGATCTACACCCGATGTAACACTC |
| | TTTCCCTACACGACGCTCT |
| 58 | AATGATACGGCGACCACCGAGATCTACACCTCGACTTACACTC |
| | TTTCCCTACACGACGCTCT |
| 59 | AATGATACGGCGACCACCGAGATCTACACGTAGTACCACACTC |
| | TTTCCCTACACGACGCTCT |
| 60 | AATGATACGGCGACCACCGAGATCTACACATTAGCCGACACTC |
| | TTTCCCTACACGACGCTCT |
| 61 | AATGATACGGCGACCACCGAGATCTACACTGGACCATACACTC |
| | TTTCCCTACACGACGCTCT |
| 62 | AATGATACGGCGACCACCGAGATCTACACCATCTGCTACACTC |
| | TTTCCCTACACGACGCTCT |
| 63 | AATGATACGGCGACCACCGAGATCTACACGACTACGAACACTC |
| | TTTCCCTACACGACGCTCT |
| 64 | AATGATACGGCGACCACCGAGATCTACACGCTTCACAACACTC |
| | TTTCCCTACACGACGCTCT |
| 65 | AATGATACGGCGACCACCGAGATCTACACAACGTAGCACACTC |
| | TTTCCCTACACGACGCTCT |
| 66 | AATGATACGGCGACCACCGAGATCTACACACCATGTCACACTC |
| | TTTCCCTACACGACGCTCT |
| 67 | AATGATACGGCGACCACCGAGATCTACACCTGTGGTAACACTC |
| | TTTCCCTACACGACGCTCT |
| 68 | AATGATACGGCGACCACCGAGATCTACACGTTGGCATACACTC |
| | TTTCCCTACACGACGCTCT |
| 69 | AATGATACGGCGACCACCGAGATCTACACGATACCTGACACTC |
| | TTTCCCTACACGACGCTCT |
| 70 | AATGATACGGCGACCACCGAGATCTACACGACGTCATACACTC |
| | TTTCCCTACACGACGCTCT |
| 71 | AATGATACGGCGACCACCGAGATCTACACCAGGATGTACACTC |
| | TTTCCCTACACGACGCTCT |
| 72 | AATGATACGGCGACCACCGAGATCTACACACACCGATACACTC |
| | TTTCCCTACACGACGCTCT |
| 73 | AATGATACGGCGACCACCGAGATCTACACTGCTTGCTACACTC |
| | TTTCCCTACACGACGCTCT |
| 74 | AATGATACGGCGACCACCGAGATCTACACTGGAAGCAACACTC |
| | TTTCCCTACACGACGCTCT |
| 75 | AATGATACGGCGACCACCGAGATCTACACTATGACCGACACTC |
| | TTTCCCTACACGACGCTCT |
| 76 | AATGATACGGCGACCACCGAGATCTACACCCGCTTAAACACTC |
| | TTTCCCTACACGACGCTCT |
| 77 | AATGATACGGCGACCACCGAGATCTACACCCTCGTTAACACTC |
| | TTTCCCTACACGACGCTCT |
| 78 | AATGATACGGCGACCACCGAGATCTACACAGCTAAGCACACTC |
| | TTTCCCTACACGACGCTCT |
| 79 | AATGATACGGCGACCACCGAGATCTACACCTAAGACCACACTC |
| | TTTCCCTACACGACGCTCT |
| 80 | AATGATACGGCGACCACCGAGATCTACACTCACCTAGACACTC |
| | TTTCCCTACACGACGCTCT |
| 81 | AATGATACGGCGACCACCGAGATCTACACGCATAACGACACTC |
| | TTTCCCTACACGACGCTCT |
| 82 | AATGATACGGCGACCACCGAGATCTACACAGGTTCCTACACTC |
| | TTTCCCTACACGACGCTCT |
| 83 | AATGATACGGCGACCACCGAGATCTACACCGAGTTAGACACTC |
| | TTTCCCTACACGACGCTCT |
| 84 | AATGATACGGCGACCACCGAGATCTACACTCTTCGACACACTC |
| | TTTCCCTACACGACGCTCT |
| 85 | AATGATACGGCGACCACCGAGATCTACACTACTGCTCACACTC |
| | TTTCCCTACACGACGCTCT |
| 86 | AATGATACGGCGACCACCGAGATCTACACCTGCCATAACACTC |
| | TTTCCCTACACGACGCTCT |
| 87 | AATGATACGGCGACCACCGAGATCTACACCCAAGTAGACACTC |
| | TTTCCCTACACGACGCTCT |
| 88 | AATGATACGGCGACCACCGAGATCTACACGACCGATAACACTC |
| | TTTCCCTACACGACGCTCT |
| 89 | AATGATACGGCGACCACCGAGATCTACACCATACGGAACACTC |
| | TTTCCCTACACGACGCTCT |
| 90 | AATGATACGGCGACCACCGAGATCTACACTCTAGTCCACACTC |
| | TTTCCCTACACGACGCTCT |
| 91 | AATGATACGGCGACCACCGAGATCTACACAGTGACCTACACTC |
| | TTTCCCTACACGACGCTCT |
| 92 | AATGATACGGCGACCACCGAGATCTACACACCTAGACACACTC |
| | TTTCCCTACACGACGCTCT |
| 93 | AATGATACGGCGACCACCGAGATCTACACGTGGTATGACACTC |
| | TTTCCCTACACGACGCTCT |
| 94 | AATGATACGGCGACCACCGAGATCTACACGTTATGGCACACTC |
| | TTTCCCTACACGACGCTCT |
| 95 | AATGATACGGCGACCACCGAGATCTACACAACAGCGAACACTC |
| | TTTCCCTACACGACGCTCT |
| 96 | AATGATACGGCGACCACCGAGATCTACACGTCCTGTTACACTC |
| | TTTCCCTACACGACGCTCT |

TABLE 8

| Pair | Reverse Primer (SEQ. ID. No: 1001-196) |
|---|---|
| 1 | CAAGCAGAAGACGGCATACGAGATGCGAATTGGTGACTGGAGT |
| | TCAGACGTGTGC |
| 2 | CAAGCAGAAGACGGCATACGAGATAACCAGAGGTGACTGGAGT |
| | TCAGACGTGTGC |
| 3 | CAAGCAGAAGACGGCATACGAGATAATGCCGAGTGACTGGAGT |
| | TCAGACGTGTGC |
| 4 | CAAGCAGAAGACGGCATACGAGATCGAAGCTTGTGACTGGAGT |
| | TCAGACGTGTGC |
| 5 | CAAGCAGAAGACGGCATACGAGATGGACAGTAGTGACTGGAGT |
| | TCAGACGTGTGC |
| 6 | CAAGCAGAAGACGGCATACGAGATTACGTGCTGTGACTGGAGT |
| | TCAGACGTGTGC |
| 7 | CAAGCAGAAGACGGCATACGAGATACTGAAGCGTGACTGGAGT |
| | TCAGACGTGTGC |
| 8 | CAAGCAGAAGACGGCATACGAGATTGCCTAAGGTGACTGGAGT |
| | TCAGACGTGTGC |
| 9 | CAAGCAGAAGACGGCATACGAGATTCGTGAAGGTGACTGGAGT |
| | TCAGACGTGTGC |
| 10 | CAAGCAGAAGACGGCATACGAGATAGTTCCTGGTGACTGGAGT |
| | TCAGACGTGTGC |
| 11 | CAAGCAGAAGACGGCATACGAGATGTGTGTACGTGACTGGAGT |
| | TCAGACGTGTGC |
| 12 | CAAGCAGAAGACGGCATACGAGATCTTCTCCAGTGACTGGAGT |
| | TCAGACGTGTGC |
| 13 | CAAGCAGAAGACGGCATACGAGATACTAGCCTGTGACTGGAGT |
| | TCAGACGTGTGC |
| 14 | CAAGCAGAAGACGGCATACGAGATCTCTATGCGTGACTGGAGT |
| | TCAGACGTGTGC |
| 15 | CAAGCAGAAGACGGCATACGAGATGCAACGTTGTGACTGGAGT |
| | TCAGACGTGTGC |
| 16 | CAAGCAGAAGACGGCATACGAGATCGGATAGTGTGACTGGAGT |
| | TCAGACGTGTGC |
| 17 | CAAGCAGAAGACGGCATACGAGATCCTGACAAGTGACTGGAGT |
| | TCAGACGTGTGC |
| 18 | CAAGCAGAAGACGGCATACGAGATCACAAGGAGTGACTGGAGT |
| | TCAGACGTGTGC |
| 19 | CAAGCAGAAGACGGCATACGAGATGTACTCCTGTGACTGGAGT |
| | TCAGACGTGTGC |
| 20 | CAAGCAGAAGACGGCATACGAGATAGACGAGAGTGACTGGAGT |
| | TCAGACGTGTGC |
| 21 | CAAGCAGAAGACGGCATACGAGATGAAGCAAGGTGACTGGAGT |
| | TCAGACGTGTGC |
| 22 | CAAGCAGAAGACGGCATACGAGATCTCGAGTTGTGACTGGAGT |
| | TCAGACGTGTGC |
| 23 | CAAGCAGAAGACGGCATACGAGATCTAAGTCGGTGACTGGAGT |
| | TCAGACGTGTGC |
| 24 | CAAGCAGAAGACGGCATACGAGATGCGTTAGAGTGACTGGAGT |
| | TCAGACGTGTGC |
| 25 | CAAGCAGAAGACGGCATACGAGATGGTTCAACGTGACTGGAGT |
| | TCAGACGTGTGC |
| 26 | CAAGCAGAAGACGGCATACGAGATACGTGGTTGTGACTGGAGT |
| | TCAGACGTGTGC |
| 27 | CAAGCAGAAGACGGCATACGAGATACCATGTCGTGACTGGAGT |
| | TCAGACGTGTGC |
| 28 | CAAGCAGAAGACGGCATACGAGATGAGTCTCAGTGACTGGAGT |
| | TCAGACGTGTGC |
| 29 | CAAGCAGAAGACGGCATACGAGATTGATCCTCGTGACTGGAGT |
| | TCAGACGTGTGC |
| 30 | CAAGCAGAAGACGGCATACGAGATCATAACCGGTGACTGGAGT |
| | TCAGACGTGTGC |
| 31 | CAAGCAGAAGACGGCATACGAGATTCTACTCCGTGACTGGAGT |
| | TCAGACGTGTGC |
| 32 | CAAGCAGAAGACGGCATACGAGATTGTCGAGTGTGACTGGAGT |
| | TCAGACGTGTGC |
| 33 | CAAGCAGAAGACGGCATACGAGATCTACTCTGGTGACTGGAGT |
| | TCAGACGTGTGC |
| 34 | CAAGCAGAAGACGGCATACGAGATACGCGAATGTGACTGGAGT |
| | TCAGACGTGTGC |
| 35 | CAAGCAGAAGACGGCATACGAGATACCATCGAGTGACTGGAGT |
| | TCAGACGTGTGC |
| 36 | CAAGCAGAAGACGGCATACGAGATGTCACTTCGTGACTGGAGT |
| | TCAGACGTGTGC |
| 37 | CAAGCAGAAGACGGCATACGAGATCATGAGAGGTGACTGGAGT |
| | TCAGACGTGTGC |
| 38 | CAAGCAGAAGACGGCATACGAGATAGATTGCGGTGACTGGAGT |
| | TCAGACGTGTGC |
| 39 | CAAGCAGAAGACGGCATACGAGATGTAACGGAGTGACTGGAGT |
| | TCAGACGTGTGC |
| 40 | CAAGCAGAAGACGGCATACGAGATCAACATCGGTGACTGGAGT |
| | TCAGACGTGTGC |
| 41 | CAAGCAGAAGACGGCATACGAGATGGATGACTGTGACTGGAGT |
| | TCAGACGTGTGC |
| 42 | CAAGCAGAAGACGGCATACGAGATTCCGTATCGTGACTGGAGT |
| | TCAGACGTGTGC |
| 43 | CAAGCAGAAGACGGCATACGAGATAGTGTCGTGTGACTGGAGT |
| | TCAGACGTGTGC |
| 44 | CAAGCAGAAGACGGCATACGAGATTAGAAGCCGTGACTGGAGT |
| | TCAGACGTGTGC |
| 45 | CAAGCAGAAGACGGCATACGAGATGTTCCAGAGTGACTGGAGT |
| | TCAGACGTGTGC |
| 46 | CAAGCAGAAGACGGCATACGAGATGTTCGTCAGTGACTGGAGT |
| | TCAGACGTGTGC |
| 47 | CAAGCAGAAGACGGCATACGAGATCGGTGAATGTGACTGGAGT |
| | TCAGACGTGTGC |
| 48 | CAAGCAGAAGACGGCATACGAGATTGTAACGCGTGACTGGAGT |
| | TCAGACGTGTGC |
| 49 | CAAGCAGAAGACGGCATACGAGATTTGCCAGTGTGACTGGAGT |
| | TCAGACGTGTGC |
| 50 | CAAGCAGAAGACGGCATACGAGATTAAGCTCCGTGACTGGAGT |
| | TCAGACGTGTGC |
| 51 | CAAGCAGAAGACGGCATACGAGATCATCTCGTGTGACTGGAGT |
| | TCAGACGTGTGC |
| 52 | CAAGCAGAAGACGGCATACGAGATCATTGCTGGTGACTGGAGT |
| | TCAGACGTGTGC |
| 53 | CAAGCAGAAGACGGCATACGAGATGGCTGATAGTGACTGGAGT |
| | TCAGACGTGTGC |
| 54 | CAAGCAGAAGACGGCATACGAGATAGGACGATGTGACTGGAGT |
| | TCAGACGTGTGC |
| 55 | CAAGCAGAAGACGGCATACGAGATGAAGGCATGTGACTGGAGT |
| | TCAGACGTGTGC |
| 56 | CAAGCAGAAGACGGCATACGAGATGTTGTGGAGTGACTGGAGT |
| | TCAGACGTGTGC |
| 57 | CAAGCAGAAGACGGCATACGAGATATGTAGCCGTGACTGGAGT |
| | TCAGACGTGTGC |
| 58 | CAAGCAGAAGACGGCATACGAGATTTCAGCTCGTGACTGGAGT |
| | TCAGACGTGTGC |
| 59 | CAAGCAGAAGACGGCATACGAGATCCATGATGGTGACTGGAGT |
| | TCAGACGTGTGC |
| 60 | CAAGCAGAAGACGGCATACGAGATGCCGATTAGTGACTGGAGT |
| | TCAGACGTGTGC |
| 61 | CAAGCAGAAGACGGCATACGAGATTACCAGGTGTGACTGGAGT |
| | TCAGACGTGTGC |
| 62 | CAAGCAGAAGACGGCATACGAGATTCGTCTACGTGACTGGAGT |
| | TCAGACGTGTGC |
| 63 | CAAGCAGAAGACGGCATACGAGATAGCATCAGGTGACTGGAGT |
| | TCAGACGTGTGC |
| 64 | CAAGCAGAAGACGGCATACGAGATACACTTCGGTGACTGGAGT |
| | TCAGACGTGTGC |
| 65 | CAAGCAGAAGACGGCATACGAGATCGATGCAAGTGACTGGAGT |
| | TCAGACGTGTGC |
| 66 | CAAGCAGAAGACGGCATACGAGATCTGTACCAGTGACTGGAGT |
| | TCAGACGTGTGC |
| 67 | CAAGCAGAAGACGGCATACGAGATATGGTGTCGTGACTGGAGT |
| | TCAGACGTGTGC |
| 68 | CAAGCAGAAGACGGCATACGAGATTACGGTTGGTGACTGGAGT |
| | TCAGACGTGTGC |
| 69 | CAAGCAGAAGACGGCATACGAGATGTCCATAGGTGACTGGAGT |
| | TCAGACGTGTGC |
| 70 | CAAGCAGAAGACGGCATACGAGATTACTGCAGGTGACTGGAGT |
| | TCAGACGTGTGC |
| 71 | CAAGCAGAAGACGGCATACGAGATTGTAGGACGTGACTGGAGT |
| | TCAGACGTGTGC |
| 72 | CAAGCAGAAGACGGCATACGAGATTAGCCACAGTGACTGGAGT |
| | TCAGACGTGTGC |
| 73 | CAAGCAGAAGACGGCATACGAGATTCGTTCGTGTGACTGGAGT |
| | TCAGACGTGTGC |
| 74 | CAAGCAGAAGACGGCATACGAGATACGAAGGTGTGACTGGAGT |
| | TCAGACGTGTGC |
| 75 | CAAGCAGAAGACGGCATACGAGATGCCAGTATGTGACTGGAGT |
| | TCAGACGTGTGC |
| 76 | CAAGCAGAAGACGGCATACGAGATAATTCGCCGTGACTGGAGT |
| | TCAGACGTGTGC |
| 77 | CAAGCAGAAGACGGCATACGAGATATTGCTCCGTGACTGGAGT |
| | TCAGACGTGTGC |
| 78 | CAAGCAGAAGACGGCATACGAGATCGAATCGAGTGACTGGAGT |
| | TCAGACGTGTGC |
| 79 | CAAGCAGAAGACGGCATACGAGATCCAGAATCGTGACTGGAGT |
| | TCAGACGTGTGC |
| 80 | CAAGCAGAAGACGGCATACGAGATGATCCACTGTGACTGGAGT |
| | TCAGACGTGTGC |
| 81 | CAAGCAGAAGACGGCATACGAGATGCAATACGGTGACTGGAGT |
| | TCAGACGTGTGC |
| 82 | CAAGCAGAAGACGGCATACGAGATTCCTTGGAGTGACTGGAGT |
| | TCAGACGTGTGC |
| 83 | CAAGCAGAAGACGGCATACGAGATGATTGAGCGTGACTGGAGT |
| | TCAGACGTGTGC |
| 84 | CAAGCAGAAGACGGCATACGAGATCAGCTTCTGTGACTGGAGT |
| | TCAGACGTGTGC |
| 85 | CAAGCAGAAGACGGCATACGAGATCTCGTCATGTGACTGGAGT |
| | TCAGACGTGTGC |
| 86 | CAAGCAGAAGACGGCATACGAGATATACCGTCGTGACTGGAGT |
| | TCAGACGTGTGC |
| 87 | CAAGCAGAAGACGGCATACGAGATGATGAACCGTGACTGGAGT |
| | TCAGACGTGTGC |
| 88 | CAAGCAGAAGACGGCATACGAGATATAGCCAGGTGACTGGAGT |
| | TCAGACGTGTGC |
| 89 | CAAGCAGAAGACGGCATACGAGATAGGCATACGTGACTGGAGT |
| | TCAGACGTGTGC |
| 90 | CAAGCAGAAGACGGCATACGAGATCCTGATCTGTGACTGGAGT |
| | TCAGACGTGTGC |
| 91 | CAAGCAGAAGACGGCATACGAGATTCCAGTGAGTGACTGGAGT |
| | TCAGACGTGTGC |
| 92 | CAAGCAGAAGACGGCATACGAGATCAGATCCAGTGACTGGAGT |
| | TCAGACGTGTGC |
| 93 | CAAGCAGAAGACGGCATACGAGATGTATGGTGGTGACTGGAGT |
| | TCAGACGTGTGC |
| 94 | CAAGCAGAAGACGGCATACGAGATCGGTATTGGTGACTGGAGT |
| | TCAGACGTGTGC |
| 95 | CAAGCAGAAGACGGCATACGAGATAGCGACAAGTGACTGGAGT |
| | TCAGACGTGTGC |
| 96 | CAAGCAGAAGACGGCATACGAGATTTGTCCTGGTGACTGGAGT |
| | TCAGACGTGTGC |

In one aspect, it will be appreciated that embodiments of modular nucleic acid adapters may include any combination of the features described herein. In one example, the scheme illustrated in Table 5 contemplates adapters having UIDs with a length of 2 nucleotides and variable length punctuation marks, whereas the scheme illustrated in Table 6 contemplates adapters having UIDs with a length of 2 nucleotides and single nucleotide punctuation marks (i.e., the punctuation marks are not of a variable lengths).

The present application is not to be limited in scope by the specific embodiments described herein. Indeed, various modifications in addition to those described herein will become apparent to those skilled in the art from the foregoing description and accompanying figures. Such modifications are intended to fall within the scope of the claims. Various publications are cited herein, the disclosures of which are incorporated by reference in their entireties.

1. A kit for preparing a library of nucleic acids having adapter sequences for sequencing, the kit comprising:
a first oligonucleotide having a first tail sequence, a first common sequence, and at least one of i) a first unique identifier sequence, and ii) a first variable length punctuation mark;
a second oligonucleotide having a second tail sequence, a second common sequence complimentary to the first common sequence, and at least one of i) a second unique identifier sequence complimentary to the first unique identifier sequence, and ii) a second variable length punctuation mark complimentary to the first variable length punctuation mark;
a first primer having a first sample identifier sequence and a first priming sequence at a 3′ end of the first primer, the first priming sequence including the first tail sequence of the first oligonucleotide; and
a second primer having a second sample identifier sequence and a second priming sequence at a 3′ end of the second primer, the second priming sequence being complimentary to the second tail sequence of the second oligonucleotide.

2. The kit of claim 1, wherein the first sample identifier sequence and the second sample identifier sequence have a one-to-one mapping.

3. The kit of claim 2, wherein the first variable length punctuation mark has a length of 2-4 nucleotides.

4. The kit of claim 2, where the first variable length punctuation mark includes at least one of a G and a C nucleotide.

5. The kit of claim 1, wherein the first unique identifier sequence has a length of at least 5 nucleotides.

6. The kit of claim 5, wherein the first unique identifier sequence has a pairwise edit distance of at least 3.

7. A kit for preparing a library of nucleic acids having adapter sequences for sequencing, the kit comprising:
a plurality of oligonucleotide pairs, each of the oligonucleotide pairs including:
a first oligonucleotide having a first tail sequence, a first common sequence, and at least one of i) a first unique identifier sequence, and ii) a first variable length punctuation mark, and
a second oligonucleotide having a second tail sequence, a second common sequence complimentary to the first common sequence, and at least one of i) a second unique identifier sequence complimentary to the first unique identifier sequence, and ii) a second variable length punctuation mark complimentary to the first variable length punctuation mark,
a first primer having a first sample identifier sequence and a first priming sequence at a 3′ end of the first primer, the first priming sequence including the first tail sequence of the first oligonucleotide; and
a second primer having a second sample identifier sequence and a second priming sequence at a 3′ end of the second primer, the second priming sequence being complimentary to the second tail sequence of the second oligonucleotide.

8. The kit of claim 7, wherein each of the first unique identifier sequences of each of the plurality of oligonucleotide pairs is different.

9. The kit of claim 7, wherein each of the first tail sequences of each of the plurality of oligonucleotide pairs is the same.

10. The kit of claim 7, wherein each of the second tail sequences of each of the plurality of oligonucleotide pairs is the same.

11. The kit of claim 7, wherein each of the plurality of oligonucleotide pairs are annealed to form a forked adapter.

12. The kit of claim 7, wherein the first sample identifier sequence and the second sample identifier sequence have a one-to-one mapping.

13. The kit of claim 7, wherein each of the first unique identifier sequences has a length of at least 5 nucleotides.

14. The kit of claim 15, wherein each of the first unique identifier sequences has a pairwise edit distance of at least 3.

15. A method of preparing a library of nucleic acid molecules, the method comprising:
attaching one of a plurality of oligonucleotide adapters to each end of a target nucleic acid to provide an adapter-target-adapter construct, each of the plurality of oligonucleotide adapters having:
a first oligonucleotide having a first tail sequence, a first common sequence, and at least one of i) a first unique identifier sequence, and ii) a first variable length punctuation mark, and
a second oligonucleotide having a second tail sequence, a second common sequence complimentary to the first common sequence, and at least one of i) a second unique identifier sequence complimentary to the first unique identifier sequence, and ii) a second variable length punctuation mark complimentary to the first variable length punctuation mark;
annealing a first primer to the adapter-target-adapter construct, the first primer having a first sample identifier sequence and a first priming sequence at a 3′ end of the first primer, the first priming sequence including the first tail sequence of the first oligonucleotide; and
extending each of the first primer and the second primer to form extension products complementary to each strand of the adapter-target-adapter constructs.
